(19) United States
(12) Patent Application Publication
(10) Pub. No.: US 2021/0299281 A1
(43) Pub. Date: Sep. 30, 2021

(54) MULTIPLE BIOMARKERS IMAGING FOR HIGH SPECIFICITY

(71) Applicant: Board of Regents, The University of Texas System, Austin, TX (US)

(72) Inventor: Baohong Yuan, Arlington, TX (US)

(21) Appl. No.: 17/260,759

(22) PCT Filed: Jul. 17, 2019
(86) PCT No.: PCT/US2019/042119

§ 371 Date: Jan. 15, 2021

Related U.S. Application Data
(60) Provisional application No. 62/699,490, filed on Jul. 17, 2018.

(51) Int. Cl. A61K 49/00; G01N 33/574; G01N 33/542; G01N 33/58; A61K 49/22; A61B 5/00; G01N 21/64

(52) U.S. Cl. CPC A61K 49/0002; G01N 33/57492; G01N 33/542; G01N 33/582; A61K 49/0093; G01N 2021/6441; A61K 49/222; A61K 49/223; A61B 5/0097; A61B 5/489; G01N 21/6428; A61K 49/0091

(57) ABSTRACT

A method of imaging cancer stem cells comprises disposing a population of first ultrasound-switchable fluorophorms having a first switching threshold in the biological environment, the first ultrasound-switchable fluorophores being functionalized for attachment to a first biomarker expressed by the CSCs; disposing a population of second ultrasound-switchable fluorophorms having a second switching threshold in the biological environment, the second ultrasound-switchable fluorophores being functionalized for attachment to a second biomarker expressed by the CSCs; exposing the biological environment to an ultrasound beam to form an activation region; disposing one or more of the first and/or second ultrasound-switchable fluorophores in the activation region to switch the first and/or second fluorophores from an off state to an on state; exciting the first and second ultrasound-switchable fluorophores in the activation region with a beam of electromagnetic radiation; and detecting light emitted by the first and second ultrasound-switchable fluorophores.

### CROSS-REFERENCE TO RELATED APPLICATIONS

This application claims priority pursuant to 35 U.S.C. § 119(e) to U.S. Provisional Patent Application No. 62/699,490, filed on Jul. 17, 2018, which is hereby incorporated by reference in its entirety.

### STATEMENT REGARDING GOVERNMENT FUNDING

This invention was made with government support under contract CBET-1253199 awarded by the National Science Foundation, and under contract RP170564 awarded by the Cancer Prevention and Research Institute of Texas (CPRIT). The government has certain rights in the invention.

### FIELD

The invention is generally related to diagnostic imaging of cancer stem cells, and, more specifically, to multi-color ultrasound-switchable fluorescence microscopy for cancer stem cell imaging.

### BACKGROUND

Many malignant tumors contain tumor-initiating cells, known as cancer stem cells (“CSCs”), that account for <1-3% of the overall cancer cells. CSCs are believed to function as the seeds of malignant tumors and have been observed to self-renew. CSCs have also been observed as a source of cancer treatment resistance, because CSCs are not as active as regular cancer cells and insensitive to conventional treatments. Survived CSCs can lead to cancer relapse and metastasis, where cancer relapse means that cancer grows again after the primary cancer (such as a tumor) was surgically removed or treated. Cancer relapse is unfortunately common, and current CSC theory believes that CSCs are the seeds of most malignant cancers. If even small amounts of CSCs are unintentionally left during a surgery or other treatments, the probability of cancer relapse is high.

Unfortunately, CSCs are usually insensitive and resist to conventional therapies (such as radio- and chemotherapies). This is because conventional therapies are usually designed for non-CSCs (the main part of a solid tumor). These non-CSCs usually display high metabolic activity and rapid proliferation, so that they are sensitive to the conventional therapeutic drugs or radiations and can be killed. However, CSCs are usually metabolically “quiet” and in a silent state. Thus, conventional treatments do not efficiently target CSCs. Therefore, it is common to see a tumor significantly shrink after treatments (i.e. non-CSCs are killed), but later a new tumor re-grows (CSCs survive and function as seeds to regrow). If the location of CSCs before, during, and/or after a surgery or therapy can be identified, appropriate treatment strategies for CSCs can be developed and applied to remove most (or all) of CSCs present in a tissue, reducing the rate of cancer relapse.

One approach to identifying CSCs location is through diagnostic imaging. While this approach is conventionally used to identify non-CSCs, conventional imaging methods do not work well or at all for identifying CSCs. CSCs is much more challenging than imaging non-CSCs because: (1) the amount of CSCs is much smaller, requiring a more sensitive technology; (2) CSCs are mixed with non-CSCs, requiring higher specificity to CSCs; (3) the size of CSC-rich regions is much smaller, requiring high resolution; and (4) CSCs are often deeply located in tissue, requiring large imaging depths. None of current imaging technologies can simultaneously satisfy all these requirements.

Many different conventional technologies have been explored to image CSCs: magnetic resonance imaging (MRI), position emission tomography (PET), single-photon emission computed tomography (SPECT), fluorescence molecular imaging/tomography (FMI/FMT), bioluminescence imaging (BLI), intravital fluorescence microscopy (IFM), photoacoustic imaging (PAI), among others. While these conventional technologies display varying degrees of success in imaging non-CSCs, all of them fail to reliably image CSCs for a variety of reasons. For example, all of these CSC imaging technologies are limited to imaging one biomarker at a time, and lack multiplex imaging capability for simultaneous imaging of multiple biomarkers (SIMB). MRI, CT, PET and ultrasound are less attractive for SIMB because encoding their radiation is difficult. Optical absorption spectra of photoacoustic contrast agents are too wide to be able to efficiently differentiate different agents, and PAI signal is not specific to its contrast agents because blood and water may generate strong noise (due to the large volume). While NIR fluorescence molecular tomography and SPECT theoretically are capable of SIMB via spectroscopic methods, they both display poor spatial resolution (˜3-5 mm) in centimeters deep tissue. Without good spatial resolution in deep tissue, differentiation of multiple biomarkers meaningless because the spectroscopically differentiated biomarkers would be completely mixed again in the spatial domain.

In addition to these problems, these conventional imaging modalities are limited by their fundamental physics. MRI and CT are limited by their low sensitivity to contrast agents. PET/SPECT, FMI/FMT and BLI are limited by poor spatial resolution. IFM is only for superficial tissue (<1 mm). Ultrasound microbubbles cannot reach CSCs, because the large size of microbubbles (˜2-3 microns) confine the agents in blood vessels. PAI is limited by its moderate sensitivity and specificity (blood generates large background noise due to its large volume).

Therefore, there is a need for improved imaging systems and methods for identifying CSCs that simultaneously is sensitive enough to detect small amounts of CSCs, can differentiate CSCs from non-CSCs, have high resolution that can image very small CSC-rich regions, and have sufficiently deep imaging depth in tissue.

### SUMMARY

Methods of imaging CSCs in a biological environment are described herein which, in some cases, can provide one or more advantages compared to other methods. For example, in some embodiments, methods of imaging CSCs in a biological environment are described herein using near infrared (NIR) ultrasound-switchable fluorescence microscopy (USFM) for simultaneous imaging of multiple biomarkers (SIMCB). USFM has multiplex imaging capability via multi-spectral (i.e. multi-color) fluorescence emission, and can simultaneously image multiple biomarkers of CSCs to specifically differentiate CSCs from non-CSCs. USFM has high sensitivity (picomoles or better) because of high photon detection sensitivity, allowing for exclusive differentiation of signal photons from background noise photons. USFM can also image as deep as centimeters with high resolution, allowing imaging of CSCs populations in a variety of tissues, such as breast, prostate, head and neck, thyroid, oral, skin, colon, uterus, and other tissues. Moreover, USFM is inexpensive and uses non-ionizing radiations (ultrasound and light), which enables for repeated and longitudinal studies.

In an aspect, a method of imaging cancer stem cells (CSCs) in a biological environment comprises disposing a population of first ultrasound-switchable fluorophores having a first switching threshold in the biological environment, the first ultrasound-switchable fluorophores being functionalized for attachment to a first biomarker expressed by the CSCs; disposing a population of second ultrasound-switchable fluorophores having a second switching threshold in the biological environment, the second ultrasound-switchable fluorophores being functionalized for attachment to a second biomarker expressed by the CSCs; exposing the biological environment to an ultrasound beam to form an activation region within the biological environment; disposing one or more of the first ultrasound-switchable fluorophores in the activation region to switch the first fluorophores from an off state to an on state; disposing one or more of the second ultrasound-switchable fluorophores in the activation region to switch the second fluorophores from an off state to an on state; exciting the first and second ultrasound-switchable fluorophores in the activation region with a beam of electromagnetic radiation; and detecting light emitted by the first and second ultrasound-switchable fluorophores.

The first or second ultrasound-switchable fluorophores can comprise a fluorescent material having a peak emission wavelength between 680 nm and 710 nm. In some cases, the first or second ultrasound-switchable fluorophores comprise a fluorescent material having a peak emission wavelength between 740 nm and 770 nm. In some embodiments, the first or second ultrasound-switchable fluorophores comprises a fluorescent material having a peak emission wavelength that is longer than 800 nm. In further embodiments, the first or second ultrasound-switchable fluorophores comprises a fluorescent material having an emission tail that is longer than 900 nm. In some embodiments, the first ultrasound-switchable fluorophores emit light having a first average peak wavelength and the second ultrasound-switchable fluorophores emit light having a second average peak wavelength, and wherein the second average peak wavelength is 25-75 nm longer than the first average peak wavelength.

In some embodiments, exposing the biological environment to an ultrasound beam comprises scanning the biological environment with the ultrasound beam. In some cases, the beam of electromagnetic radiation is in the near-infrared region (NIR) of the electromagnetic spectrum. The first and second ultrasound-switchable fluorophores in the activation region can be excited by a single beam of electromagnetic radiation in some instances.

The biological environment can comprise tumor vasculature in some embodiments.

In some embodiments, the first and second ultrasound-switchable fluorophores comprise a thermo-sensitive polymer, such as poly(N-isopropylacrylamide), a copolymer of N-isopropylacrylamide with one or more of acrylamide, N-tert-butylacrylamide, acrylic acid, and allylamine, or a polyoxypropylene-polyoxyethylene block copolymer.

In some embodiments, the method further comprises disposing a population of third ultrasound-switchable fluorophores having a third switching threshold in the biological environment, the third ultrasound-switchable fluorophores being functionalized for attachment to a third bio-target expressed by the CSCs; disposing one or more of the third ultrasound-switchable fluorophores in the activation region to switch the third fluorophores from an off state to an on state; exciting the first, second, and third ultrasound-switchable fluorophores in the activation region with the beam of electromagnetic radiation; and detecting light emitted by the first, second, and third ultrasound-switchable fluorophores.

In some instances, the first ultrasound-switchable fluorophores emit light having a first average peak wavelength; the second ultrasound-switchable fluorophores emit light having a second average peak wavelength; the third ultrasound-switchable fluorophores emit light having a third average peak wavelength; and the third average peak wavelength is longer than each of the first and second average peak wavelengths. The light emitted by the first, second, and third pluralities of fluorophores can be simultaneously detected, and the first, second, and third switching thresholds are temperature thresholds.

In another aspect, a method of imaging a tumor comprises disposing first ultrasound-switchable fluorophores having a first switching threshold temperature in the tumor, the first ultrasound-switchable fluorophores being associated with a first emission spectrum; disposing second ultrasound-switchable fluorophores having a second switching threshold temperature in the tumor, the second ultrasound-switchable fluorophores being associated with a second emission spectrum; moving an ultrasound beam over a plurality of spatial locations within a plane of the tumor, wherein a temperature of the tumor within a focal zone of the ultrasound beam exceeds the first and second switching threshold temperatures thereby switching the first and second fluorophores in the focal zone from an off state to an on state; exciting the first and second ultrasound-switchable fluorophores in the focal zone with a beam of electromagnetic radiation; detecting light emitted by the first and second ultrasound-switchable fluorophores; and characterizing a spatial location as a possible CSC location in response to detecting light having peak wavelengths in each of the first and second emission spectra, and characterizing the spatial location as a non-CSC location in response to detecting light having a peak wavelength in only one of the first or the second emission spectrum.

In some embodiments, the plane of the tumor is located at a depth of 1-6 centimeters (cm) below a surface of a subject's skin. In some instances, the tumor is located in a subject's breast, prostate, head, neck, throat, mouth, thyroid, skin, colon, cervix, or uterus

The method can further comprises generating a multi-colored image of the tumor, wherein spatial locations that correspond to emissions in the first emission spectra are correlated to a first color, spatial locations that correspond to emissions in the second emission spectra are correlated to a second color, and spatial locations that correspond to emissions being a combination of the first and second emission spectra are correlated to a third color.

These and other embodiments are described in greater detail in the detailed description which follows.

### BRIEF DESCRIPTION OF THE DRAWINGS

The invention will now be described by way of example, with reference to the following drawings, of which:

FIG. 1 illustrates a thermal switching event of an ultrasound-switchable fluorophores;

FIGS. 2(a) and 2(b) illustrate schematically steps of a method of forming an activation region;

FIG. 3(a) is a plot of excitation profiles for a series of ultrasound-switchable fluorophores;

FIG. 3(b) is a plot of emission profiles for a series of ultrasound-switchable fluorophores;

FIG. 4(a) illustrates schematically a multi-color USFM imaging system;

FIG. 4(b) is a graphical illustration of time sequence of firing three pico-second (ps) pulsed lasers and the gating time of three cooled-and-gated intensified charge-coupled devices (CG-ICCD);

FIG. 5 illustrates an exemplary USF contrast agent bound to a microbubble and to a targeting moiety through biotin-streptavidin linkages;

FIG. 6 illustrates the structure of a carboxylated nano-capsule (USFM contrast agent) used for conjugation with antibodies of CSC biomarkers;

FIG. 7 illustrates an exemplary CSC cell bound with three different USFM contrast agents having different CSC biomarker antibodies and different fluorophores;

FIG. 8 is a graphical illustration of the in vivo pharmacokinetics of three-color USFM contrast agents;

FIG. 9 is a USF image of two different USF contrast agents being differentiated via different excitation and emission spectra;

FIG. 10 is a graph of emission spectrum of a near-infrared II USF contrast agent at two different temperatures; and

FIG. 11 is a USF image of a near-infrared II USF contrast agent in centimeter-deep tissue.

### DETAILED DESCRIPTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

The terms “first,” “second,” “third,” and so on, are to be understood as generally distinguishing one object from another, rather than referring to a quantity of that object. For example, when describing three separate objects, each object can be distinguished from the two objects by the names “first object,” “second object,” and “third object”. In cases where a quantity of an object is being described, the description with particularly identify that a quantity of the object is being discussed.

In an aspect, methods of imaging cancer stem cells (CSCs) in a biological environment are described herein. In some embodiments, the method comprises disposing a population of first ultrasound-switchable fluorophores having a first switching threshold in the biological environment, the first ultrasound-switchable fluorophores being functionalized for attachment to a first biomarker expressed by the CSCs (a first targeting ultrasound-switchable fluorophore); disposing a population of second ultrasound-switchable fluorophores having a second switching threshold in the biological environment, the second ultrasound-switchable fluorophores being functionalized for attachment to a second biomarker expressed by the CSCs (a second targeting ultrasound-switchable fluorophore); exposing the biological environment to an ultrasound beam to form an activation region within the biological environment; disposing one or more of the first ultrasound-switchable fluorophores in the activation region to switch the first fluorophores from an off state to an on state; disposing one or more of the second ultrasound-switchable fluorophores in the activation region to switch the second fluorophores from an off state to an on state; exciting the first and second ultrasound-switchable fluorophores in the activation region with a beam of electromagnetic radiation; and detecting light (e.g. photoluminescence signals) emitted by the first and second ultrasound-switchable fluorophores.

It is also possible to use a third targeting ultrasound-switchable fluorophore. For example, the method of imaging CSCs in a biological environment can further comprise disposing a population of third ultrasound-switchable fluorophores having a third switching threshold in the biological environment, the third ultrasound-switchable fluorophores being functionalized for attachment to a third bio-target (i.e. biomarker) expressed by the CSCs; disposing one or more of the third ultrasound-switchable fluorophores in the activation region to switch the third fluorophores from an off state to an on state; exciting the first, second, and third ultrasound-switchable fluorophores in the activation region with the beam of electromagnetic radiation; and detecting light (e.g. photoluminescence signals) emitted by the first, second, and third ultrasound-switchable fluorophores. In an embodiment, the first, second, and third switching thresholds are temperature thresholds, as discussed in more detail below.

The method of imaging cancer stem cells (CSCs) in a biological environment is not limited to three targeting ultrasound-switchable fluorophores. More generally, n different targeting ultrasound-switchable fluorophores can be used. Thus, in some embodiments, the method can comprise disposing a population of n ultrasound-switchable fluorophores having a n switching thresholds in the biological environment, each of the n ultrasound-switchable fluorophores being functionalized for attachment to n bio-targets expressed by the CSCs (n targeting ultrasound-switchable fluorophores); exposing the biological environment to an ultrasound beam to form an activation region within the biological environment; disposing the n targeting ultrasound-switchable fluorophores in the activation region to switch the n fluorophores from an off state to an on state; exciting n ultrasound-switchable fluorophores in the activation region with a beam of electromagnetic radiation; and detecting light emitted by the n ultrasound-switchable fluorophores. It is further to be understood that n can be any desired integer, such as 2, 3, 4, 5, 6, 7, 8, 9, or 10. Moreover, in some cases, the number n is selected based on a desired number of biomarkers being targeted (targeting elements) in the method.

In some instances, the first ultrasound-switchable fluorophores are configured to emit light having a first average peak wavelength; the second ultrasound-switchable fluorophores are configured to emit light having a second average peak wavelength; the third ultrasound-switchable fluorophores are configured to emit light having a third average peak wavelength; where the third average peak wavelength is longer than each of the first and second average peak wavelengths.

In another aspect, a method of imaging a tumor is disclosed, the method comprising disposing first ultrasound-switchable fluorophores having a first switching threshold temperature in the tumor, the first ultrasound-switchable fluorophores being associated with a first emission spectrum; disposing second ultrasound-switchable fluorophores having a second switching threshold temperature in the tumor, the second ultrasound-switchable fluorophores being associated with a second emission spectrum; moving an ultrasound beam over a plurality of spatial locations within a plane of the tumor, wherein a temperature of the tumor within a focal zone of the ultrasound beam exceeds the first and second switching threshold temperatures thereby switching the first and second fluorophores in the focal zone from an off state to an on state; exciting the first and second ultrasound-switchable fluorophores in the focal zone with a beam of electromagnetic radiation; detecting light emitted by the first and second ultrasound-switchable fluorophores; and characterizing a spatial location as a possible cancer stem cell (CSC) location in response to detecting light having peak wavelengths in each of the first and second emission spectra, and characterizing the spatial location as a non-CSC location in response to detecting light having a peak wavelength in only one of the first or the second emission spectrum.

Again, in some instances, the method of imaging a tumor can further comprise disposing n ultrasound-switchable fluorophores having a n switching threshold temperatures in the tumor, each n ultrasound-switchable fluorophore being associated with an nth emission spectra; moving an ultrasound beam over a plurality of spatial locations within a plane of the tumor, wherein a temperature of the tumor within a focal zone of the ultrasound beam exceeds the n switching threshold temperatures thereby switching the n fluorophores in the focal zone from an off state to an on state; exciting the n ultrasound-switchable fluorophores in the focal zone with a beam of electromagnetic radiation; detecting light emitted (e.g. photoluminescence) by the n ultrasound-switchable fluorophores; and characterizing a spatial location as a possible cancer stem cell (CSC) location in response to detecting light having peak wavelengths in each of the nth emission spectra, and characterizing the spatial location as a non-CSC location in response to detecting light having a peak wavelength in only one of thein emission spectrum.

In some embodiments, the method of imaging a tumor further comprises generating a multi-colored image of the tumor, wherein spatial locations that correspond to emissions in the first emission spectra are correlated to a first color, spatial locations that correspond to emissions in the second emission spectra are correlated to a second color, and spatial locations that correspond to emissions being a combination of the first and second emission spectra are correlated to a third color.

In some embodiments, the photoluminescent emission signals of individual ultrasound-switchable fluorophores described herein are individually resolved or isolated. Such individual resolution or isolation can be achieved by selectively exciting a given ultrasound-switchable fluorophore and/or by selectively detecting the resulting emission of the given ultrasound-switchable fluorophore. For example, in some cases, the beam of electromagnetic radiation has a wavelength maximum sufficient to simultaneously excite the first, second, and/or third targeting fluorophores in the on state. Moreover, in other cases, exposing the environment to a beam of electromagnetic radiation comprises exposing the environment to first and second beams of electromagnetic radiation (which can be referred to as a first excitation beam and a second excitation beam, respectively), wherein the first excitation beam and the second excitation beam have different wavelength maximums. This can be particularly desirable when the first and second ultrasound-switchable fluorophores have differing absorption or excitation profiles. For example, in some embodiments, the first excitation beam can primarily excite the first targeting ultrasound-switchable fluorophore, and the second excitation beam can primarily excite the second non-targeting ultrasound-switchable fluorophore at a different wavelength maximum. This principle also applies when n targeting fluorophores are used, where the environment is exposed to n excitation beams, each having different wavelength maximums. The first, second, and n excitation beams can be provided to the environment sequentially or non-simultaneously. In some instances, the first, second, and n excitation beams are provided to the environment simultaneously.

In some such embodiments, the first photoluminescence signal is primarily associated with or correlated to photoluminescence of the first targeting ultrasound-switchable fluorophore, and the second photoluminescence signal is primarily associated with or correlated to photoluminescence of the second non-targeting ultrasound-switchable fluorophore. Further, in some such embodiments, the first and second photoluminescence signals are detected using different detectors or detection channels, and the detectors or detection channels are turned on or off in accordance with the sequence of the excitation beams, such that a given detector or detection channel is operable to receive and process a photoluminescence signal (i.e., is “on”) or not (i.e., is “off”) only or primarily only from a desired fluorophore, namely, the fluorophore primarily excited by the desired excitation beam. For instance, in some cases, the first photoluminescence signal is detected with a first detector or detection channel, and the second photoluminescence signal is detected with a second detector or detection channel. Moreover, the first detector or detection channel is off when the second photoluminescence signal is detected, and the second detector or detection channel is off when the first photoluminescence signal is detected. In other embodiments, the first detector or detection channel and the second detector or detection channel are on at the same time, and simultaneously detect their respective photoluminescence signals.

It is to be understood that the foregoing principle can be extended to n ultrasound-switchable fluorophores, n photoluminescence signals, and n detectors or detection channels. For instance, in some embodiments, exposing the environment to a beam of electromagnetic radiation comprises exposing the environment to n excitation beams, wherein the n excitation beams have differing wavelength maximums, wherein the nth excitation beam primarily excites the nth ultrasound-switchable fluorophore, and wherein the n excitation beams are provided to the environment sequentially or non-simultaneously. Moreover, in some such cases, n photoluminescent signals are detected with n differing detectors or detection channels, and each nth photoluminescence signal is primarily associated with or correlated to photoluminescence of the nth ultrasound-switchable fluorophore. Further, the n detectors or detection channels are turned on or off in accordance with the sequence of the excitation beams, such that each nth detector or detection channel is on when the nth photoluminescence signal is to be detected (or when the nth fluorophore is primarily excited) and off when the nth photoluminescence signal is not to be detected (or when the nth fluorophore is not primarily excited). As described above, n can be any desired integer, such as 2, 3, 4, 5, 6, 7, 8, 9, or 10. For example, when n=3, the light emitted by the first, second, and third plurality of fluorophores can be simultaneously detected.

Moreover, it is further to be understood that it is also possible for n ultrasound-switchable fluorophores to be used, but for less than n detectors or detection channels (and/or less than n excitation beams or excitation sources) to be used (provided n is at least 2). In such instances, the detectors or detection channels (and/or excitation beams or excitation sources) can be shared and/or used in an alternating or other temporally resolved manner.

Resolution or isolation of individual photoluminescence signals primarily associated with individual ultrasound-switchable fluorophores can be further achieved or improved, if needed, by using excitation and/or emission filters for one or more excitation beams and/or one or more detectors or detection channels, as described further hereinbelow. It is also possible to deconvolute or resolve individual photoluminescence signals primarily associated with individual ultrasound-switchable fluorophores by carrying out one or more signal processing steps, as described further hereinbelow and/or as known to one of ordinary skill in the art. For example, in some cases, multivariate curve resolution is used, as described in Xu et al., “In-vivo fluorescence imaging with a multivariate curve resolution spectral unmixing technique,” J. Biored. Opt. 14 (2009).

Turning now to specific steps of methods, methods described herein comprise disposing a population of ultrasound-switchable fluorophores in an environment. Any environment not inconsistent with the objectives of the current disclosure may be used. In some embodiments, the environment is a biological environment. An environment of a method described herein may also be a non-biological environment. In some cases, for example, a biological environment is an in vivo environment, such as a tissue, organ, blood vessel, or other portion of a living organism. In some embodiments, the biological environment comprises a tumor or tumor vasculature. The tumor or tumor vasculature can be located in any tissue or organ in a living organism, such as breast, prostate, head, neck, throat, mouth, thyroid, skin, colon, cervix, or uterus. In other cases, a biological environment comprises an in vitro environment, such as a tissue culture. The biological environment of a method described herein can also comprise or be replaced by a biological phantom material or tissue-mimicking phantom material, such as an agar, silicone, polyvinyl alcohol (PVA) gel, polyacrylamide (PAA) gel, or a dispersion of an oil in gelatin. Other phantom materials may also be used.

Moreover, in some embodiments, a biological environment comprises deep tissue. “Deep” tissue, for reference purposes herein, comprises tissue (or, in the case of a phantom material, an interior region of the phantom material) that is located at least about 1 cm below the exterior or outer surface of the organism, tissue culture, or other larger structure associated with the biological environment (such as, in the case of a phantom material, the outer surface of the phantom material). In some embodiments, for instance, deep tissue is located between about 1 cm and about 10 cm, between about 1 cm and about 6 cm, or between about 1 cm and about 5 cm below an outer surface. In some cases, deep tissue is located more than 10 cm below an outer surface. Further, an outer surface, in some embodiments, comprises the surface of the skin of an organism.

In addition, any ultrasound-switchable fluorophore or combination of differing ultrasound-switchable fluorophores not inconsistent with the objectives of the current disclosure may be used. An “ultrasound-switchable” fluorophore, for reference purposes herein, comprises a fluorophore operable to switch between an on state and an off state in response to exposure to an ultrasound beam. The ultrasound beam can be either directly or indirectly responsible for the switching response of the fluorophore. For example, in some cases, the ultrasound beam interacts directly with the fluorophore, resulting in a switch between fluorescence states of the fluorophore. In other cases, the ultrasound beam interacts directly with the immediate environment or microenvironment of the fluorophore and changes at least one property of the fluorophore's microenvironment. In such cases, the fluorophore can switch between on and off fluorescence states in response to the environmental change induced by the ultrasound beam. A non-limiting example of an environmental change would be a change in temperature. Thus, the fluorophore can be indirectly switchable in response to exposure to an ultrasound beam.

The “on” state of a fluorophore, for reference purposes herein, comprises either (1) a state at which the fluorescence intensity of the fluorophore is relatively high compared to the “off” state of the fluorophore, at which the fluorescence intensity is relatively low (assuming the fluorophore is similarly excited in both the on state and the off state); or (2) a state at which the fluorescence lifetime of the fluorophore is relatively long compared to the “off” state of the fluorophore, at which the fluorescence lifetime is relatively short (again assuming the fluorophore is similarly excited). Further, in both cases, the on and off states substantially define a step function in the fluorescence intensity or lifetime profile when plotted as a function of a critical switching parameter such as temperature. A fluorophore having a longer lifetime in an on state than an off state can be particularly suitable for use in methods described herein using time-gated or time-delayed detection of emitted photons from fluorophores, such as time-gated detection in which only those photons received after a relatively long delay following excitation are counted by the detector as part of the USF signal. In some cases, the on state of a fluorophore exhibits at least about 70 percent, at least about 80 percent, or at least about 90 percent of the theoretical maximum fluorescence intensity of the fluorophore, and the off state of the fluorophore exhibits no more than about 50 percent, no more than about 30 percent, no more than about 10 percent, or no more than about 5 percent of the theoretical maximum fluorescence intensity of the fluorophore.

The physical cause for the existence of an on state versus an off state can vary. For example, in some cases, the fluorescence intensity or fluorescence lifetime of a fluorophore changes dues to a conformational or chemical change of the fluorophore in response to a change in environmental conditions, such as exhibited by some thermoresponsive polymers, pH-sensitive chemical species, or pressure sensitive materials. In some cases, the fluorescence intensity or fluorescence lifetime of a fluorophore changes in response to internal fluorescence quenching, wherein such quenching can be directly or indirectly induced by the presence of ultrasound.

For example, in some embodiments, a fluorophore described herein comprises a Förster resonance energy transfer (FRET) donor species and a FRET acceptor species, and the distance between the FRET donor species and the FRET acceptor species is altered by the presence of an ultrasound beam. The FRET donor species can be a first fluorescent species or other chromophore, and the FRET acceptor species can be a second fluorescent species or other chromophore. In such cases, as understood by one of ordinary skill in the art, FRET energy transfer between the donor species and the acceptor species can result in quenching of the fluorescence of the donor species. Thus, the acceptor species can be considered to be a fluorescence quenching species of the fluorophore. Any donor-acceptor pair not inconsistent with the objectives of the current disclosure may be used in FRET-based fluorophores described herein. For example, in some cases, the donor species comprises Alexa Fluor 546 and the acceptor species comprise Alexa Fluor 647. Other combinations of acceptor species and donor species are also possible.

In some embodiments, a fluorophore described herein comprises a microbubble comprising one or more FRET donor species and one or more FRET acceptor species attached to the exterior surface of the microbubble, wherein the microbubble is operable to change in size in response to the presence of an ultrasound beam. The change in size can increase or decrease the distance between the FRET donor species and the FRET acceptor species, thus reducing or increasing the FRET energy transfer efficiency. As a result, the fluorescence quenching and the overall fluorescence intensity of the microbubble can vary based on the size of the microbubble.

A microbubble described herein can have any size and be formed of any chemical species not inconsistent with the objectives of the current invention. In some cases, a microbubble has a diameter between about 1 μm and about 10 μm or between about 1 μm and about 5 μm. Other sizes of microbubbles may also be used. Moreover, in some embodiments, a microbubble described herein comprises a gas core surrounded by a shell formed from a polymeric material, such an organic polymeric material. In other cases, the shell is formed from a lipid material. In some embodiments, a microbubble comprises a shell formed from one or more of albumin, galactose, lipid, and sulfur hexafluoride. In addition, the gas core of a microbubble described herein can comprise one or more of air, nitrogen, and a perfluorocarbon such as octafluoropropane. Moreover, in some cases, a microbubble described herein is formed from a commercially available microbubble, such as a SonoVue™, Optison™, Imagent™, Definity™, or Targestar™ microbubble. A FRET donor and/or acceptor species described herein can be attached to the surface of such a microbubble in any manner not inconsistent with the objectives of the current invention. In some cases, for instance, a donor and/or acceptor species is attached to the exterior surface of a commercially available microbubble using one or more of a carbodiimide, maleimide, or biotin-streptavidin coupling scheme. Moreover, any other coupling scheme not inconsistent with the objectives of the current disclosure can be used to attach a donor and/or acceptor species to a microbubble.

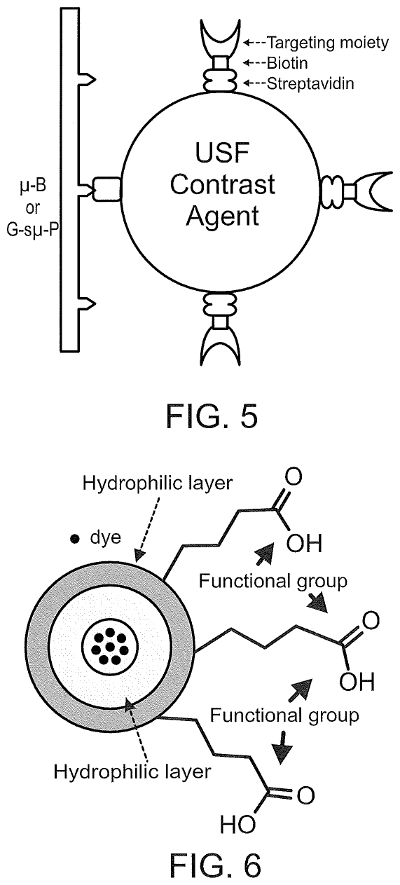

In an embodiment, gas-filled micro-particles, such as the above described microbubbles, generate a short but high temperature pulse in and around the particle surface when the microbubble is irradiated with an ultrasound pulse at diagnostic intensity level. This short temperature pulse spatially decays very fast (only ˜0.2° C. left at a distance of 1 micron away from the bubble surface). In ultrasound imaging, tissue overheating caused by microbubbles is minimalized from this fast temperature decay. However, this microscopic heating principle is effective for heating ultrasound switchable fluorophores, because ultrasound switchable fluorophores are small nanoparticles that can be attached on the microbubble's surface. As seen for example in FIG. 5, ultrasound switchable fluorophores (e.g. USF Contrast Agents) can be attached to a microbubble via a biotin/streptavidin linkage. Moreover, any other linkage not inconsistent with the objectives of the current disclosure can be used to attach ultrasound switchable fluorophores to a microbubble.

In other embodiments, a highly ultrasound-absorbing polymer, biodegradable polyurethane with pendent carboxyl groups (PU-COOH), can alternatively be used instead of the microbubbles. These ultrasound-absorbing polyurethanes make relatively rigid gas-filled sub-micro-particles (˜700 nm in diameter). For example, in some embodiments, the ultrasound-absorbing polymer can comprise a Pluronic polymer with pendent carboxyl groups similar in size to the polyurethanes, such as F127, F98, F98-PEG20k, F98-PEG30k, F98-PEG40k, F68 and its PEGylated polymers, which have been functionalized to incorporate pendent carboxyl groups. These ultrasound-absorbing polymers are generally smaller in diameter than microbubbles, reducing their acoustic attenuation compared to microbubbles. However, their relatively rigid structures can sometimes display more resilient bio-stability than microbubbles. Similar to the microbubbles, biotin can be incorporated onto the surface of the ultrasound-absorbing polymers, and the USF contrast agents can be attached using the streptavidin linkage. Moreover, any other coupling scheme not inconsistent with the objectives of the current disclosure can be used to attach a donor and/or acceptor species to a microbubble.

In some embodiments, a fluorophore described herein comprises a thermoresponsive polymer. A “thermoresponsive” polymer, for reference purposes herein, comprises a polymer having a physical or chemical property that changes in a temperature-dependent manner, wherein the change is a discontinuous or binary change. For example, in some cases, the physical conformation or polarity of a thermoresponsive polymer changes in a temperature-dependent manner, and the thermoresponsive polymer exhibits a first conformation below a threshold temperature and a second, substantially different conformation above the threshold temperature. In some embodiments, for instance, a thermoresponsive polymer exhibits an expanded coil or chain confirmation below a threshold temperature and exhibits a compact or globular conformation above the threshold temperature. In some such cases, the threshold temperature can be referred to as the “lower critical solution temperature” (LCST) of the polymer.

Any thermoresponsive polymer not inconsistent with the objectives of the current invention may be used. In some embodiments, a thermoresponsive polymer comprises a poly(N-isopropylacrylamide) or a copolymer of N-isopropylacrylamide with one or more of acrylamide, N-tert-butylacrylamide, acrylic acid, allylamine, or a polyoxypropylene-polyoxyethylene block copolymer. In other cases, a thermoresponsive polymer comprises a poly(N-vinylcaprolacatam) (PVCL) or a poloxamer such as a Pluronic polymer. Other thermoresponsive polymers may also be used.

Additionally, in some cases, a thermoresponsive polymer of a fluorophore described herein comprises one or more fluorescent moieties or is conjugated to one or more fluorescent species, such as one or more fluorescent dye molecules. The fluorescent dye molecules can comprise any fluorescent dyes not inconsistent with the objectives of this disclosure, such as the commercially available ZnPC (Zinc phthalocyanines) family of dyes (e.g. ZnPc, ZnPcTTB, ZnPcHF, ZnPcOB, among others), the ADP(CA)₂ family of dyes, or ICG-based agents (indocyanine greens, including ICG-encapsulated agents such as ICG-encapsulated poly(N-isopropylacrylamide) (PNIPAM)). The thermoresponsive polymer can be conjugated to the fluorescent species in any manner not inconsistent with the objectives of the current invention. For example, in some cases, a thermoresponsive polymer is coupled to a fluorescent species through one or more covalent bonds such as one or more ester bonds or one or more amide bonds.

Some non-limiting examples of an ultrasound-switched fluorescence process using a thermoresponsive fluorophore are illustrated in U.S. Patent Application Publication No. 2015/0309014 to Yuan et al. (hereinafter “the '014 publication”), which is incorporated herein in its entirety. As described in the '014 publication, a thermoresponsive polymer can be conjugated to a fluorescent species to provide a fluorophore. The fluorophore has a chain conformation and a globular conformation described hereinabove, and the conformation is temperature-dependent. Further, the transition from one conformation to the other results in a change in the fluorescence intensity or lifetime of the fluorescent species. As described further herein, the change in fluorescence intensity or lifetime can be due to differences in the microenvironment of the fluorescent species when the polymer is in the chain conformation compared to the globular conformation. For example, in some cases, the polarity and/or viscosity of the polymer environment experienced by the fluorophore changes depending on whether the polymer is in the chain conformation or the globular conformation.

Further, in some embodiments, a fluorophore described herein comprises a fluorescent material dispersed in and/or attached to the surface of a thermoresponsive polymer nanoparticle. Moreover, the fluorescence properties of the fluorescent material can be dependent on a change of the conformation, polarity, or other physical or chemical property of the polymer nanoparticle. In addition, the property change can be a temperature-dependent change. In this manner, a change in temperature of the thermoresponsive polymer nanoparticle can result in a change in fluorescence intensity and/or lifetime of the fluorescent material, including a change between an on state of the fluorescent material and an off state of the fluorescent material.

For example, in some embodiments, a thermoresponsive polymer nanoparticle can exhibit a temperature-dependent polarity, and the fluorescent material dispersed in the nanoparticle can exhibit a polarity-dependent fluorescence intensity and/or lifetime. Thus, a change in the temperature of the nanoparticle can result in a change in the fluorescence intensity and/or lifetime of the fluorophore.

In another exemplary embodiment, a thermoresponsive polymer nanoparticle can have a hydrophilic interior below a threshold temperature and a hydrophobic interior above the threshold temperature. Thus, such a nanoparticle can exhibit a temperature-dependent size when dispersed in a polar or non-polar solvent. For example, when dispersed in water or another polar solvent below the threshold temperature, the nanoparticle can exhibit a larger size due to the presence of water in the hydrophilic interior of the nanoparticle. Similarly, above the threshold temperature, the nanoparticle can exhibit a smaller size due to the exclusion of water from the now hydrophobic interior of the nanoparticle. In this manner, a fluorescent material dispersed in the nanoparticle can have a temperature-dependent concentration, which can result in temperature-dependent fluorescence properties of the overall fluorophore. This process is illustrated schematically in the '014 publication, specifically in FIG. 2.

In another embodiment, an ultrasound-switchable fluorophore is formed by incorporating a fluorescent material such as a fluorescent dye within the interior of a polymeric nanoparticle or micelle, such that the polymeric nanoparticle or micelle acts as a nanocapsule for the fluorescent material. Moreover, the polymeric nanoparticle can be formed from a thermoresponsive polymer, such as a thermoresponsive polymer described hereinabove. Non-limiting examples of polymers suitable for forming nanocapsules described herein include Pluronic F127, F98, F98-PEG20k, F98-PEG30k, F98-PEG40k, F68 and its PEGylated polymers, poly(N-isopropylacrylamide) or a copolymer of N-isopropylacrylamide with one or more of acrylamide, N-tert-butylacrylamide, acrylic acid, allylamine, or a polyoxypropylene-polyoxyethylene block copolymer, or poly(N-vinylcaprolacatam) (PVCL). Moreover, in some instances, a nanoparticle or nanocapsule can be formed by copolymerizing a thermoresponsive polymer described hereinabove with a polyethylene glycol (PEG) and/or by conjugating a PEG as a pendant group to a thermoresponsive polymer. Such a fluorophore, in some cases, can have a switching threshold that is controlled at least in part by the inclusion of PEG, as described further in the '014 publication.

A polymer nanoparticle such as a thermoresponsive polymer nanoparticle or a polymer nanocapsule described herein can have any size or shape not inconsistent with the objectives of the current disclosure. In some embodiments, for instance, a thermoresponsive polymer nanoparticle is substantially spherical and has a diameter between about 10 nm and about 300 nm, between about 50 nm and about 250 nm, between about 50 nm and about 200 nm, or between about 70 nm and about 150 nm. In some cases, a polymer nanocapsule is substantially spherical and has a diameter of less than about 100 nm or less than about 50 nm. In some instances, a polymer nanocapsule has a size between about 20 nm and about 90 nm, between about 20 nm and about 80 nm, or between about 20 nm and about 70 nm. Other sizes and shapes are also possible.

Further, any fluorescent material not inconsistent with the objectives of the current invention may be dispersed in and/or attached to a thermoresponsive polymer nanoparticle or other polymer nanoparticle to form a fluorophore described herein. In some embodiments, as described herein, the fluorescent material exhibits a polarity-sensitive fluorescence intensity and/or lifetime. In other cases, the fluorescent material exhibits a temperature-dependent, viscosity-dependent, pH-dependent, and/or an ionic strength-dependent fluorescence intensity and/or lifetime.

Non-limiting examples of fluorescent materials suitable for use in some embodiments described herein include organic dyes such as N,N-dimethyl-4-benzofurazansulfonamide (DBD); 4-(N,N-dimethylaminosulfonyl)-7-(2-aminoethylamino)-2,1,3-benzoxadiazole (DBD-ED); indocyanine green (ICG); a Dylight-700 such as Dylite-700-2B; IR-820; 3,3′-Diethylthiatricarbocyanine iodide (DTTCI); LS-277; LS-288; a cypate; a rhodamine dye such as rhodamine 6G or rhodamine B; or a coumarin. In some instances, a fluorescent material comprises an azadipyrromethene. In addition, in some cases, a fluorescent material comprises an inorganic species such as a semiconductor nanocrystal or quantum dot, including a II-VI semiconductor nanocrystal such as ZnS or CdSe or a III-V semiconductor nanocrystal such as InP or InAs. In other instances, a fluorescent material comprises a Lanthanide species. Additional non-limiting examples of fluorescent materials suitable for use in an ultrasound-switchable fluorophore described herein include the fluorescent materials described in Amin et al., “Syntheses, Electrochemistry, and Photodynamics of Ferrocene-Azadipyrromethane Donor-Acceptor Dyads and Triads,” J. Phys. Chem. A 2011, 115, 9810-9819; Bandi et al., “A Broad-Band Capturing and Emitting Molecular Triad: Synthesis and Photochemistry,” Chem. Commun., 2013, 49, 2867-2869; Jokic et al., “Highly Photostable Near-Infrared Fluorescent pH Indicators and Sensors Based on BF₂-Chelated Tetraarylazadipyrromethane Dyes,” Anal. Chem. 2012, 84, 6723-6730; Jiang et al., “A Selective Fluorescent Turn-On NIR Probe for Cysteine,” Org. Biomol. Chem., 2012, 10, 1966-1968; and Kucukoz et al., “Synthesis, Optical Properties and Ultrafast Dynamics of Aza-boron-dipyrromethane Compounds Containing Methoxy and Hydroxy Groups and Two-Photon Absorption Cross-Section,” Journal of Photochemistry and Photobiology A: Chemistry 247 (2012), 24-29; the entireties of which are hereby incorporated by reference. Other fluorescent materials may also be used.

An ultrasound-switchable fluorophore described herein can have any fluorescence emission profile not inconsistent with the objectives of the current invention. For example, in some embodiments, a fluorophore exhibits an emission profile including visible light or centered in the visible region of the electromagnetic spectrum, such as between 450 nm and 750 nm, 500 nm and 700 nm, or 550 nm and 650. In some cases, a fluorophore exhibits an emission profile including infrared (IR) light or centered in the IR region of the electromagnetic spectrum. For example, in some instances, a fluorophore described herein exhibits an emission profile centered in the near-IR (NIR, 750 nm-1.4 μm), short-wavelength IR (SWIR, 1.4-3 μm), mid-wavelength IR (MWIR, 3-8 μm), or long-wavelength IR (LWIR, 8-15 μm). Moreover, in some embodiments, a fluorophore described herein has an emission profile overlapping with a wavelength at which water and/or biological tissue has an absorption minimum, such as a wavelength between about 700 nm and about 800 nm, about 800 nm and about 900 nm, about 900 nm and 1.1 μm, or between about 1.25 μm and about 1.35 μm. Additionally, in some cases, a population of ultrasound-switchable fluorophores described herein comprise fluorophores having differing emission profiles for purposes of multiplexed imaging. For example, in some cases, a first fluorophore of a population can emit in the NIR and a second fluorophore of the population can emit in the visible region of the electromagnetic spectrum. In some instances, a fluorophore of the population has an emission spectra in one portion of the NIR, and the second fluorophore of a population has emission spectra in a different portion of the NIR, such as in the NIR-I and/or NIR-II regions (discussed in detail below).

In some embodiments, different populations of ultrasound-switchable fluorophores described herein comprise different fluorophores having different emission profiles for purposes of multiplexed imaging. For example, an emission profile of a first population of ultrasound switchable fluorophores having a first fluorophore can be between about 680 nm and about 710 nm, and the emission profile of a second population of ultrasound switchable fluorophores having a second fluorophore can be between about 740 nm and about 770 nm. In embodiments having a third population of ultrasound switchable fluorophores having a third fluorophore, the emission profile of a third fluorophore can be >840 nm or >900 nm. These emission profiles are merely exemplary, and in some instances the first, second, or third ultrasound-switchable fluorophores comprise a fluorescent material having a peak emission wavelength between 680 nm and 710 nm; between 740 nm and 770 nm, >800 nm, or >900 nm. In some instances, the first ultrasound-switchable fluorophores are configured to emit light having a first average peak wavelength and the second ultrasound-switchable fluorophores are configured to emit light having a second average peak wavelength, and wherein the second average peak wavelength is 25-75 nm longer than the first average peak wavelength. Moreover, this general principle can be applied to embodiments where in populations of ultrasound switchable fluorophores having n fluorophores are used. For example, a third ultra-sound switchable fluorophore can be configured to emit light having a third average peal wavelength that is 25 nm to 75 nm longer than the second average peak wavelength. In this manner, multiplexed imaging can be achieved.

In addition, a targeting ultrasound-switchable fluorophore described herein comprises a targeting moiety. A “targeting moiety”, for reference purposes herein, comprises a molecule having a physical or chemical binding affinity for a target element present in the environment. In cases where the environment is biological or phantom biological, the targeting moiety can be an antibody with specificity to a biomarker present in the environment. For example, the antibodies can have specificity to angiogenic biomarkers, such as non-limiting examples of vascular endothelial growth factor receptor (VEGFR), integrin, CD105, P-selectin, or any other angiogenic biomarkers known to those of ordinary skill in the art. The antibodies can have specificity to biomarkers uniquely overexpressed in cancer stem cells (CSCs), such as monoclonal antibodies anti-CD44, anti-CD133, anti-CD117, among others. In other instances, the targeting moiety can be a small molecule, polysaccharide, polypeptide, or any other molecule known to bind to a target element present in a biological environment. In some embodiments, the targeting moiety reversibly binds to the target element. In other embodiments, the targeting moiety irreversibly binds to the target element. In some cases, for instance, the targeting moiety is attached to a targeting ultrasound-switchable fluorophore using one or more of a carbodiimide, maleimide, or biotin-streptavidin coupling scheme, as seen for example in FIG. 5. Moreover, any other coupling scheme not inconsistent with the objectives of the current disclosure can be used to attach a targeting moiety to an ultrasound-switchable fluorophore. It is to be understood that in embodiments where in targeting ultrasound-switchable fluorophores are used, each targeting ultrasound fluorophore can have a different targeting moiety.

Further, in some instances a non-targeting ultrasound-switchable fluorophore can be used, where the non-targeting ultrasound-switchable fluorophore comprises a non-targeting moiety. A “non-targeting moiety”, for reference purposes herein, comprises a molecule exhibiting similar physicochemical behavior in the environment as the targeting moiety, but lacking a physical or chemical binding affinity for the target element. Stated differently, the non-targeting moiety is a negative control for the targeting moiety. For example, when the targeting moiety is an antibody with specificity to a biomarker present in the environment, the non-targeting moiety can be an isotype-matched control antibody that lacks specificity to the biomarker, but exhibits similar physicochemical behavior in the environment. In some embodiments, the non-targeting ultrasound-switchable fluorophore can be prepared without any non-targeting moiety. Instead, the ultrasound-switchable fluorophore itself can be used as a negative control to the targeting ultrasound-switchable fluorophore. In some embodiments, the non-targeting moiety is attached to a non-targeting ultrasound-switchable fluorophore using one or more of a carbodiimide, maleimide, or biotin-streptavidin coupling scheme, as seen for example in FIG. 5. Moreover, any other coupling scheme not inconsistent with the objectives of the current disclosure can be used to attach a non-targeting moiety to an ultrasound-switchable fluorophore.

Methods described herein also comprise exposing an environment, such as a biological environment, to one or more ultrasound beams to create an activation region within the environment. In some instances, one, two, three, or n ultrasound beams are used. The ultrasound beam can have any ultrasound frequency not inconsistent with the objectives of the current disclosure. In some embodiments, an ultrasound beam comprises an oscillating sound pressure wave with a frequency of greater than about 20 kHz or greater than about 2 MHz. In some cases, an ultrasound beam described herein has a frequency of up to about 5 GHz or up to about 3 GHz. In some embodiments, an ultrasound beam has a frequency between about 20 kHz and about 5 GHz, between about 50 kHz and about 1 GHz, between about 500 kHz and about 4 GHz, between about 1 MHz and about 5 GHz, between about 2 MHz and about 20 MHz, between about 2 MHz and about 10 MHz, between about 5 MHz and about 200 MHz, between about 5 MHz and about 15 MHz, between about 200 MHz and about 1 GHz, between about 500 MHz and about 5 GHz, or between about 1 GHz and about 5 GHz.

In addition, an ultrasound beam can have any power not inconsistent with the objectives of the current disclosure. In some embodiments, for instance, an ultrasound beam has a power between about 0.1 W/cm² and about 10 W/cm², between about 0.1 W/cm² and about 5 W/cm², between about 0.5 W/cm² and about 5 W/cm², between about 1 W/cm² and about 10 W/cm², or between about 1 W/cm² and about 5 W/cm². In other cases, an ultrasound beam has a power between about 100 W/cm² and about 5000 W/cm², or between about 100 W/cm² and about 3000 W/cm². In some cases, the use of an ultrasound beam having a high power, such as a power described herein, can result in the generation of non-linear effects within the activation region. Moreover, in some embodiments, the effective size of the activation region can be reduced in this manner, leading to improved imaging resolution.

An environment can be exposed to an ultrasound beam in any manner not inconsistent with the objectives of the current disclosure. For example, in some embodiments, a biological environment is exposed to an ultrasound beam described herein for only a limited duration. In some cases, for instance, the ultrasound beam is provided to the environment for less than about 1 second or less than about 500 ms. In some embodiments, the ultrasound beam is provided to the environment for less than about 300 ms, less than about 100 ms, less than about 50 ms, or less than about 10 ms. In some cases, the ultrasound beam is provided to the environment for about 1 ms to about 1 second, about 1 ms to about 500 ms, about 1 ms to about 300 ms, about 1 ms to about 100 ms, about 1 ms to about 50 ms, about 1 ms to about 10 ms, about 10 ms to about 300 ms, about 10 ms to about 100 ms, about 10 ms to about 50 ms, or about 50 ms to about 100 ms. The use of short exposure times of a biological environment to an ultrasound beam, in some embodiments, can permit the time-gating of fluorescence signals, such that a desired USF signal can be temporally separated from one or more undesired or non-analyte fluorescence signals, such as a tissue autofluorescence signal or a signal from a randomly switched-on fluorophore.

Moreover, the ultrasound beam can be a continuous wave beam or a pulsed or modulated beam. The use of a modulated or pulsed ultrasound beam, in some embodiments, can further improve the signal to noise ratio (SNR) of a method described herein by permitting frequency-gated detection of the USF signal. For example, in some cases, a pulsed or modulated ultrasound beam provides an ultrasound exposure having a specific frequency or modulation. As a result, the corresponding USF signal can also exhibit the same specific frequency or modulation. Thus, in some such cases, a lock-in amplifier is used to increase the sensitivity of the detector to the specific frequency or modulation, thus increasing the overall sensitivity and SNR of the method. The use of a modulated ultrasound beam can also improve the temperature resolution of a method described herein, as described further hereinbelow.

In some embodiments of methods described herein, a single ultrasound beam is directed toward the environment using a single ultrasound transducer, such as a high intensity focused ultrasound (HIFU) transducer. In other instances, a plurality of ultrasound beams is directed toward the environment using a plurality of ultrasound transducers. Moreover, in some cases, a first ultrasound beam is directed toward the environment at a first angle and/or from a first direction, and a second ultrasound beam is directed toward the environment at a second angle and/or from a second direction differing from the first angle and/or direction. In some embodiments, for instance, the first and second directions are orthogonal or substantially orthogonal directions, such as directions separated by 80 to 100 degrees. In other cases, the directions are separated by less than 80 degrees or more than 100 degrees. Further, if desired, additional ultrasound beams may also be directed toward the environment from additional directions or at additional angles. In such cases, the focal zones of the beams can overlap or intersect with one another to form an activation region at the intersection of the beams. In this manner, an activation region can have a smaller volume or cross section than the focal zone or cross section of a single ultrasound beam used to generate the activation region, thereby improving imaging resolution. In some cases, for instance, the activation region has a lateral dimension and/or an axial dimension of less than about 2 mm, less than 1.5 mm, or less than about 1 mm. In some embodiments, the activation region has a lateral dimension and/or an axial dimension of less than about 700 μm or less than about 500 μm. In some embodiments, the activation region has a lateral dimension and/or an axial dimension of about 300 μm to about 2 mm, about 400 μm to about 1.5 mm, about 400 μm to about 1 mm, about 400 μm to about 700 μm, or about 400 μm to about 500 μm. In some cases, the lateral and axial dimensions both have a size recited herein, including a size below about 1 mm or below about 700 μm. Moreover, in some embodiments, the lateral and axial dimensions of the activation region are different, thereby providing a relatively anisotropic activation region. Alternatively, in other instances, the lateral and axial dimensions are substantially the same, thereby providing a relatively “square” or isotropic activation region.

An “activation region,” for reference purposes herein, comprises a region of the environment in which ultrasound-switchable fluorophores described herein are or can be switched from an off state to an on state. For example, in some cases, an activation region comprises a region of high temperature compared to other portions of the environment. Moreover, as described further herein, the size, shape, and/or other properties of the activation region can be determined by the number and/or power of the one or more ultrasound beams used to form the activation region. In some cases, for instance, the size and shape of an activation region is defined by the focal zone of a single ultrasound beam. In other cases, an activation region is defined by the overlap of the focal zones of a plurality of ultrasound beams.

A fluorophore described herein can be disposed within an activation region in any manner not inconsistent with the objectives of the current disclosure. In some cases, a fluorophore enters or is disposed within an activation region of an environment by diffusing into the activation region from an adjacent area of the environment. The fluorophore can also be disposed within an activation region directly by injection. In other instances, an activation region is created within a specific location within an environment where it is known that a fluorophore or population of fluorophores is likely to be found or may be found. For example, in some embodiments, an ultrasound beam described herein is raster scanned across or within an environment, thereby producing a plurality of activation regions in different locations within the environment in a sequential manner.

Methods described herein also comprise exposing an environment to a beam of electromagnetic radiation and/or exciting at least one fluorophore in an on state with a beam of electromagnetic radiation. A fluorophore can be excited with a beam of electromagnetic radiation in any manner not inconsistent with the objectives of the current disclosure. In some embodiments, for instance, a fluorophore is excited using a laser excitation source such as a diode laser. In other instances, a fluorophore is excited using one or more light emitting diodes (LEDs) or a broadband excitation source. Moreover, an excitation source described herein can provide any wavelength of light not inconsistent with the objectives of the current disclosure. In some embodiments, a fluorophore described herein is excited with a beam of electromagnetic radiation comprising visible light, NIR light, or IR light. In other cases, the beam of electromagnetic radiation comprises ultraviolet (UV) light. In some embodiments, a fluorophore described herein is excited with a beam of electromagnetic radiation comprising a wavelength maximum of approximately 671 nm, 730 nm, 800 nm, or 810 nm. The fluorophore can also be excited with a beam of electromagnetic radiation having a wavelength between 600 nm to 900 nm, 650 nm to 850 nm, 700 nm to 800 nm, 600 nm to 800 nm, 600 nm to 700 nm, 700 nm to 900 nm, 800 nm to 900 nm, 900 nm to 1000 nm, 1000 nm to 1100 nm, 1100 nm to 1200 nm, 1200 nm to 1300 nm, 1400 nm to 1500 nm, or 1600 nm to 1700 nm.

Methods described herein also comprise detecting a photoluminescence signal or other light emitted within an environment or within a specific location within an environment. In some embodiments, for instance, a method comprises detecting light emitted by at least one ultrasound-switchable fluorophore. Light emitted by the fluorophore can be detected in any manner not inconsistent with the objectives of the current disclosure. In some embodiments, for example, detecting light emitted by at least one fluorophore in an on state comprises detecting the light in a time-gated or frequency-gated manner, including a time-gated manner or frequency-gated manner described herein. In some cases, the light emitted by the at least one fluorophore in the on state is detected after a time delay that is longer than the fluorescence lifetime of the fluorophore in the off state or longer than the fluorescence lifetime of another species present in the biological environment. For example, in some embodiments, the light emitted by the at least one fluorophore in the on state is detected after a time delay that is longer than the autofluorescence lifetime of a non-fluorophore species present in the biological environment, such as the autofluorescence lifetime of tissue, which may be up to about 4 ns or up to about 5 ns.

In addition, the photoluminescence signals of a method described herein can be detected using any detector configuration not inconsistent with the objectives of the current disclosure. In some embodiments, for instance, a photoluminescence signal is detected using a detector comprising a plurality of optical fiber collectors coupled to a camera or photon counter, such as a charge coupled device (CCD) or a photomultiplier tube (PMT). Further, in some cases, the optical fiber collectors are spatially distributed around the environment or around a detection surface of the environment (such as skin or another exterior surface of the environment). Any desired number of optical fiber collectors can be used. In some embodiments, up to 30, up to 20, or up to 10 optical fiber collectors are used. In some cases, 4-30, 4-20, 6-30, 6-20, 8-30, 8-20, 10-30, or 10-20 optical fiber collectors are used. Other configurations are also possible.

Additionally, in some cases, a plurality of photoluminescence signals at a plurality of locations within an environment is detected by raster scanning the environment. Such raster scanning can include raster scanning of one or more ultrasound beams across or within the environment, such that the ultrasound beam sequentially generates a series of activation regions at different locations within the environment. It is also possible, in some instances, to move or scan a detector described herein from location to location within the environment. Moving or scanning a detector in such a manner can increase the detection area of the method. In other cases, a two-dimensional detector such as a charge-coupled device (CCD) image sensor or camera is used to detect photoluminescence signals at a plurality of locations simultaneously.

Methods of imaging described herein, in some embodiments specifically described below in the examples, also comprise determining a photoluminescence property of the population of non-targeting fluorophores from the photoluminescence signal emitted by the population of non-targeting fluorophores. For example, the photoluminescence property can include an emission intensity, a location in the environment where the photoluminescence signal originated, an emission color, or any other photoluminescence property. Photoluminescence properties can be determined by any method not inconsistent with this disclosure. In some cases, the photoluminescence property is determined using a photoluminescence detector that measures intensity and/or geographic location of the photoluminescence signal emitted by the population of non-targeting fluorophores.

### Example 1

### Ultrasound Switchable Fluorophores

Targeting ultrasound-switchable fluorophores suitable for use in some embodiments of methods described herein are prepared and used as follows. In one embodiment, ultrasound-switchable fluorophores suitable for use in some embodiments of methods described herein are prepared in a manner described in U.S. Patent Application Publication No. 2015/0309014 to Yuan et al. (“the '014 publication”), which again, is incorporated herein in its entirety.

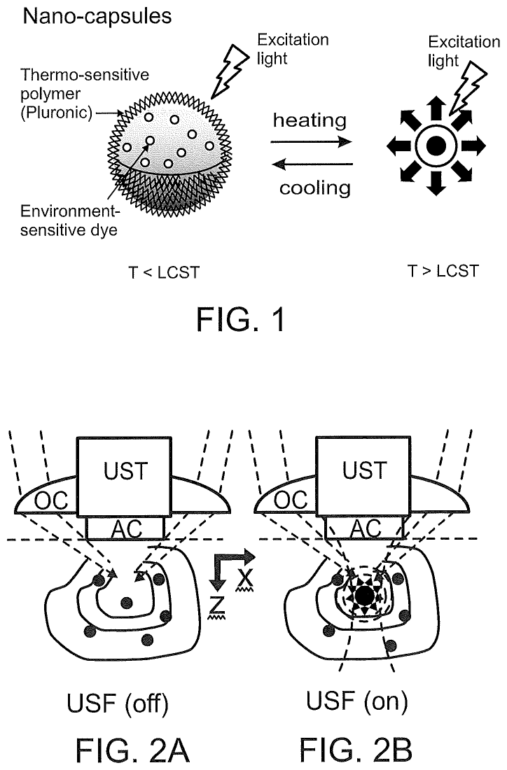

As described above, this disclosure relies generally on ultrasound fluorescence (USF or USFM) imaging. As understood by one of ordinary skill in the art and as described above, USF commonly operates according to the following principles. When an environment-sensitive near infrared (NIR) fluorescent dye (such as ZnPC, ADP(CA)₂, ICG, among others) is encapsulated into a thermo-sensitive nanoparticle (made by made by Pluronic F127, F98, F98-PEG20k, F98-PEG30k, F98-PEG40k, F68 and its PEGylated polymers, among others.), the dye's fluorescence emission exhibits a switch-like function of the temperature (FIG. 1). Briefly, when the temperature is below a threshold (T<Tₜₕ₁), the nanoparticle exhibits hydrophilicity and provides a water-rich, polar, and non-viscous microenvironment in which the dye shows very low emission efficiency (so-called OFF). When T is above another threshold (T>Tₜₕ₂), the nanoparticle exhibits hydrophobicity and provides a polymer-rich, non-polar, and viscous microenvironment in which the dye shows strong emission (so-called ON). When the transition bandwidth (T_{BW}=Tₜₕ₂−Tₜₕ₁) is narrow, the fluorescence intensity appears a switch function as the temperature. The first threshold (Tₜₕ₁) is also known as LCST (the lower critical solution temperature of the thermo-sensitive nanoparticles). In USF imaging, the threshold Ti can be controlled slightly above the tissue background temperature (T_{BG}) (i.e. T_{BG}<Tₜₕ₁) to maintain an OFF state (FIG. 2(a)). For example, Tₜₕ₁=39° C. is above T_{BG}=37° C. (body temperature). When the focused ultrasound is applied, the tissue temperature (T) at the focus will be increased above the threshold (T>Tₜₕ₁) to switch on the fluorophores (FIG. 2(b)). The USF agents outside the focus remain off. A high-resolution USF image can be formed via point-by-point scanning of ultrasound focus.

In USF imaging, NIR excitation light is delivered into centimeters deep tissue via light scattering (see the curves in FIGS. 2(a) and 2(b)). When ultrasound is off, no or weak fluorescence is emitted although the excitation light is on (see FIG. 2(a)). When ultrasound is on, the USF contrast agents in the ultrasound focal volume can be switched on to emit fluorescence (see the dashed circles in FIG. 2(b)). The emitted NIR photons can propagate out of the tissues via light scattering (towards all directions). All the ultrasound-induced fluorescence photons are signal and should be collected as many as possible. FIGS. 2(a) and 2(b) show the cross section of the sample (i.e. on x-z plane).

In USF imaging, only ultrasound-induced fluorescence photons are used as the signal. These photons can be generated only from the region around the ultrasound focus. Thus, the spatial resolution of USF depends on the size of this region. The thermal energy can be confined into the ultrasound focal region when the ultrasound exposure time is shorter enough than the thermal diffusion time (i.e. so-called thermal confinement). Unlike pure ultrasound or photoacoustic imaging (f-number usually >2), USF uses an ultrasound transducer with a small f-number (<1) to reduce the focal size. In addition, USF contrast agents can be switched on only in a region where ultrasound energy is above the switching-on threshold (Tₜₕ₁). The existence of this threshold makes the region is usually smaller than the actual size of the ultrasound-induced thermal focus. Lastly, if nonlinear acoustic effect occurs, both lateral and axial focal sizes can further shrink.

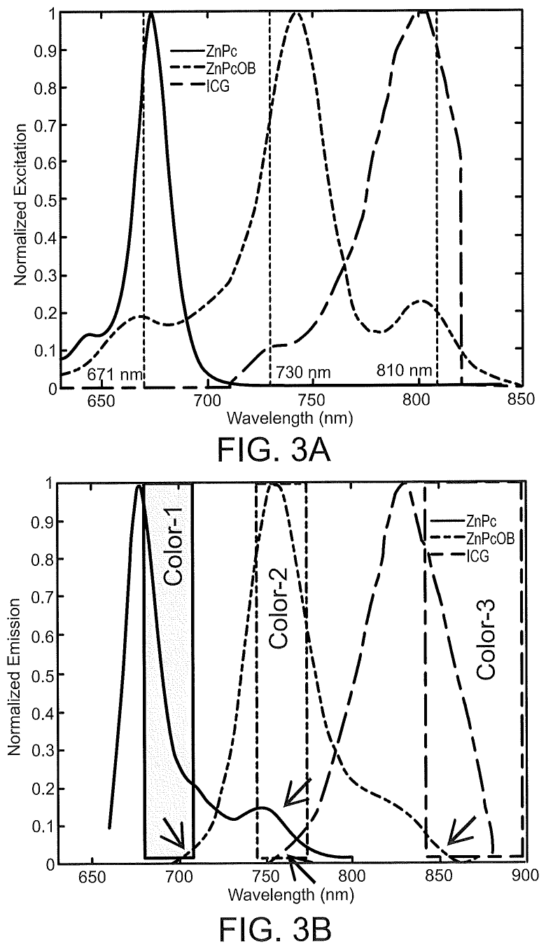

NIR-I region can cover 670-900 nm. Therefore, appropriately selecting NIR-1 fluorophores (for USF contrast agents) with different excitation (Ex) and emission (Em) wavelengths permits multi-color (multiplex) imaging to be conducted in the NIR-I region. For example, Color-1 can be selected as Ex=671 nm and Em=680-710 nm; Color-2 can be Ex=730 nm and Em=740-770 nm; and Color-3 can be Ex=810 nm and Em>840 nm (See FIGS. 3A and 3B). Although spectral cross talk may be possible, several strategies can be adopted to minimize or unmix them, as described further hereinbelow. Thus, USF can simultaneously identify multiple targets via multi-colors, which will significantly increase the specificity to the targets (none of CT, MRI, PET and ultrasound has this capability).

The NIR-II region can cover 900-1700 nm. In some embodiments, a NIR-II fluorophores (for USF contrast agents) can be selected having different excitation (Ex) and emission (Em) wavelengths that permit multi-color (multiplex) imaging to be conducted in the NIR-II region. For instance, Color 4 can be selected as Ex=800 nm and Em=912 nm. In some embodiments, a combination of both NIR-I and NIR-II fluorophores can be used.

There are two common types of spectral cross talk. The first one is so-called “one laser excites multiple fluorophores” cross talk, due to the excitation spectrum overlap. For example, when the 671-nm laser is on, it may excite both ZnPc and ZnPcOB (FIG. 3A). This type of cross talk can be reduced or avoided via sequentially turning on each laser-camera pair, as described further hereinabove and hereinbelow. Briefly, the USF system can sequentially turn on each laser-camera pair via an accurate electronic triggering system. For example, the Color-1 channel's camera is triggered ON and will detect the emission mainly from ZnPc. In contrast, the Color-2 and Color-3 channel's cameras are OFF, so the emission from ZnPcOB (excited by the 671-nm laser) will not be detected. Similarly, this rule is true for the other two laser-and-camera pairs.

The second type of cross talk is so-called “spectral bleed-through” cross talk, caused by the emission spectrum overlap. This cross talk can lead to emission leakage from one fluorophore channel to another (see the arrows in FIG. 3B). For example, when the 671-nm laser is on and possibly excites both ZnPc (strongly) and ZnPcOB (weakly), a small part of the emission from ZnPcOB (belongs to Color-2) may leak to the Color-1 channel's camera (it is the only camera that is turned on at this moment) because of the emission spectral overlap. This type of cross talk can be minimized via carefully selected emission filters and excitation light wavelengths. This type of cross talk may also be minimized or eliminated via a signal processing method, as described further herein. Also, if needed, any unavoided spectral leakage can be quantified prior to temperature measurement by using tissue phantoms and/or in vivo tissues, and then taken into account in further signal processing.

Additionally, gas-filled micro-particles, such as microbubbles, can generate a short but high temperature pulse in and around the particle surface when the microbubble is irradiated with an ultrasound pulse at diagnostic intensity level. This short temperature pulse spatially decays very fast (only ˜0.2° C. left at a distance of 1 micron away from the bubble surface). In USF imaging, tissue overheating caused by microbubbles is minimalized from this fast temperature decay. However, this microscopic heating principle is very useful for heating ultrasound switchable fluorophores, because ultrasound switchable fluorophores are small nanoparticles that can be attached on the microbubble's surface. For example, ultrasound switchable fluorophores (e.g. USF contrast agents) can be attached to a surface of a microbubble through a biotin/streptavidin linkage (see FIG. 5), or other common linkages. As seen in FIG. 5, biotin has been incorporated onto the surface of the microbubble μ-B or G-sμ-P, and streptavidin has been incorporated onto to the surface of the USF contrast agent.

A highly ultrasound-absorbing polymer, biodegradable polyurethane with pendent carboxyl groups (PU-COOH), can alternatively be used instead of the microbubbles. These ultrasound-absorbing polyurethanes make relatively rigid gas-filled sub-micro-particles that are smaller in diameter (˜700 nm in diameter) that are smaller than the microbubbles, allowing for easier transport of the attached ultrasound switchable fluorophore out of blood vessels and into CSCs. These relatively rigid particles are also much more stable than microbubbles, and their acoustic attenuation is significantly reduced because of smaller size (but still stay in blood vessels). More importantly, these particles can be efficiently heated for USF imaging because of ˜22 times higher in acoustic absorption, ˜2.3 times lower in specific heat capacity, ˜3 time lower in thermal conductivity compared with soft tissue. Similar to the microbubbles, biotin can be incorporated onto the surface of the ultrasound-absorbing polyurethanes, and the USF contrast agents can be attached using the streptavidin linkage.

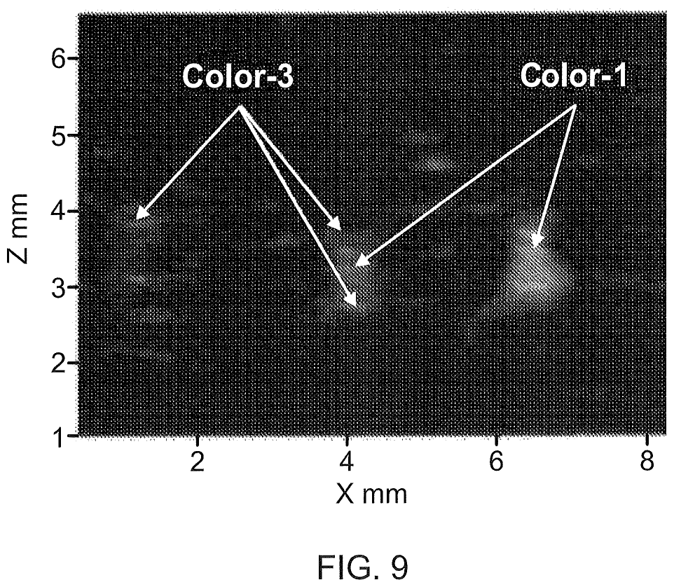

FIG. 9 shows two-color USF imaging at near infrared I (NIR-I, 650-900 nm) region, in which Color-1 represents 715 nm (emission) and Color-3 represents 830 nm (emission). In FIG. 9, three tubes were imaged using this dual-modality system, with tube 1 being on the left side of FIG. 9, tube 2 being in the middle, and tube 3 being on the right side. Tubes 1 and 3 were filled with ADP(OH)₂ (Color-3) and ICG-based USF contrast agent (Color-1), respectively. Tube 2 was filled with the two mixed solutions with a volume ratio of 3:2. To differentiate the fluorophores, ICG-based USF contrast agents were first imaged using the USF system. An 808-nm laser was used as the excitation light source. The emission filters included two 830-nm long pass interference filters and two RG830 absorption filters. The result is the fluorescence seen in tubes 2 and 3 for Color-1. No fluorescence was observed for Tube 1, because the 808-nm laser does not excite ADP(OH)₂.

To image ADP(OH)₂-based USF contrast agents in tube 1 and 2, another USF scanning was conducted using a 671-nm excitation laser and a set of emission filters (two 715-nm long pass interference filters and two RG695 absorption filters). the result is the fluorescence seen in tubes 1 and 2 for Color-3. No fluorescence was observed for Tube 3, because the 671-nm laser does not excite ICG-based USF contrast agents.

In FIG. 9, tube 2 is shown as an overlay of the two USF signals. As shown, the Color-1 fluorophore is clearly resolved from the Color-3 fluorophore, with minimal to no cross talk and emission leakage between the two fluorophores. Notably, while the image in FIG. 9 is shown in black and white, the actual image shows Tube 1 being a red color, Tube 3 being a green color, and Tube 2 being a mixture of red and green colors.

### Example 2

### Preparation of Functionalized Ultrasound Switchable Fluorophores with Prostate CSC Biomarkers

Many types of CSCs have unique expressions of biomarkers. For example, a prostate CSC can simultaneously overexpress several biomarkers such as: CD44 (a cell surface biomarker), CD133 (prominin-1, a cell surface biomarker), CD117 (c-kit, a cell surface biomarker), integrin α2β1, Sca-1, among other exemplary biomarkers. However, prostate non-CSCs cancer cells often only express one of these biomarkers. Compared with only imaging one biomarker, if multiple biomarkers of CSCs can be simultaneously differentiated and imaged, CSCs can be uniquely identified from non-CSCs and the possibility of cancer relapse and metastasis can be significantly reduced by removing the CSCs. Using the methods described herein, multiple biomarkers simultaneously labeled with multiple fluorophores can be used to differentiate and image CSCs via different excitation and emission spectra of the fluorophores (i.e. via multi-color fluorescence imaging). In contrast, non-fluorescence based imaging technologies cannot achieve this goal.

For in vitro experiments, pluronic F127 and F98 can be used because they provide relatively low switching thresholds (230 and 31° C., respectively). F127 and F98 can be used to form thermo-sensitive nano-capsules. Each nano-capsule has a hydrophilic and a hydrophobic layer and the relatively hydrophobic environment-sensitive dyes are encapsulated (FIG. 6). Because the —COOH functional groups are hydrophilic, these groups are exposed to the surrounding aqueous solution (FIG. 6). Thus, these carboxylated USFM nano-capsules can be easily conjugated on the selected monoclonal antibodies via the reaction between —COOH (on the nano-capsule) and —NH2 (on the biomarker-specific antibody). Monoclonal antibodies of CD44, CD133 and CD117 are commercially available, and can be conjugated to the carboxylated USFM nano-capsules.

For in vivo experiments, the switching threshold can be adjusted slightly above the body temperature to approximately 38°−39° C. Briefly, a hydrophilic PEG polymer with one carboxyl (—COOH) end-group and one succinimide (NHS) end-group (COOH-PEG-NHS) can be conjugated with Pluronic F98 through a coupling reaction (—COOH and —OH react to form an ester group) using the coupling agent dicyclohexylcarbodiimide (DCC). Finally, two PEG polymers can be conjugated on one Pluronic polymer to form a PEG-Pluronic-PEG polymer with two succinimide end-groups (NHS). After the formation of nano-capsules, the functional groups of NHS will be exposed to the surrounding aqueous solution for further conjugation with the functional groups of —NH₂ on the antibodies.

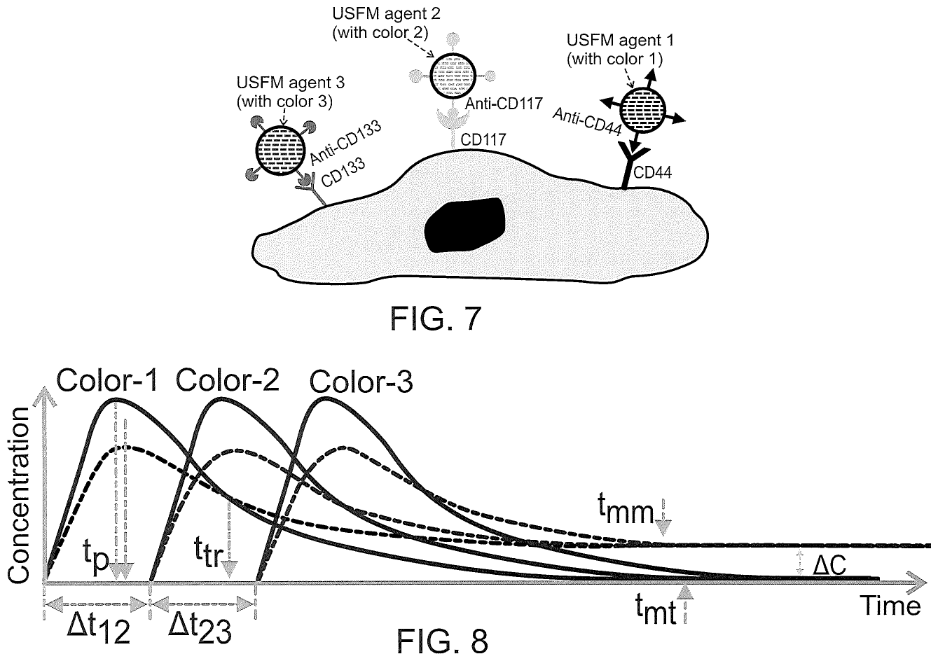

To increase the specificity, each monoclonal antibody (anti-CD44, anti-CD133, anti-CD117) can be conjugated with a USFM agent with a specific color (ZnPc, ZnPcTTB, or ZnPcHF: Color-1; ZnPcOB: Color-2; ICG-based agent: Color-3). These color-coded antibodies each target their own antigen (biomarkers) on a CSC, which in this case can be CD44, CD133 and CD 117. Thus, the specificity is correlated with the number of the detected biomarkers (i.e. the colors) and the possibility of having CSCs at each location can be calculated. When all three biomarkers are simultaneously detected, they are mostly likely from CSCs. If any colors are absent, the particular cell is likely a non-CSCs or background. FIG. 7 shows this concept and an algorithm to calculate the possibility of existing CSCs at each location is described below.

### Example 3

### Multi-Color USF System

Multi-color USF systems suitable for use in some embodiments of methods described herein are provided as follows. Unlike conventional USF systems that can only sequentially image tissue with two colors via two sequential scans, the multi-color USF system described herein can simultaneously image three colors, such as the three CSCs biomarkers described above, in a single scan. The systems generally include optical, electronic and acoustic subsystems. The time to fire a laser (excitation) pulse and the time to trigger its corresponding camera is accurately controlled. Also, the optical and electronic systems are synchronized with a high-intensity focused ultrasound (HIFU) therapeutic system for simultaneous thermal treatment and temperature monitoring. Moreover, the system comprises an algorithm to estimate the CSCs existence probability based on the measured data, to eliminate potential effects of tissue herogeneity and optical property variation at different wavelengths (the colors).

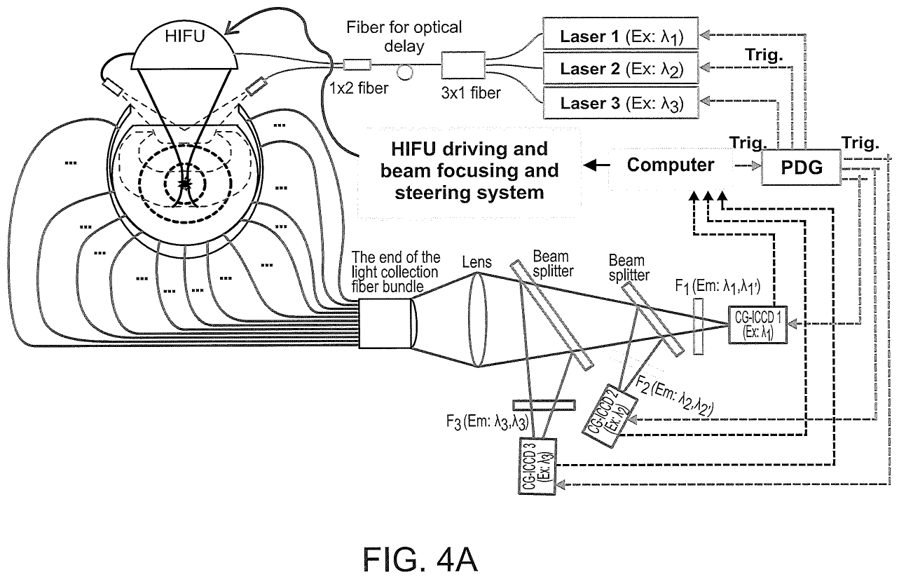

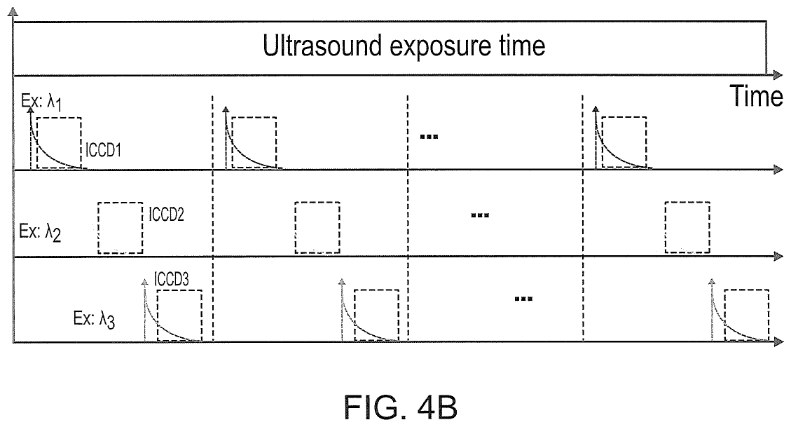

A new sensitive, three-color USFM system is shown in FIG. 4a that can simultaneously image three or more colors in a single scan. Briefly, the sample or animal is placed into a cavity. To couple the acoustic wave from the HIFU transducer into the sample or animal, an acoustic coupler is be positioned between the transducer and the surface of the tissue. Alternatively, the cavity is filled with an ultrasound-coupling medium that is optically clear, such as water. Generally, the liquid is warmed to a temperature that matches the animal body temperature. The animal is wrapped with a thin and transparent film to avoid direct contact with the medium. The mouse nose is exposed out of the medium and covered with anesthesia nose cone. Note that USFM imaging (˜minutes) is much faster than using HIFU to treat tissue (˜hours). Therefore, animals are not in the liquid (if the matching liquid is adopted) longer than 20 minutes.

Using the prostate as an example (since the above described biomarkers are prostate CSCs-specific) conventionally available transrectal ultrasound or HIFU probe is used by modifying the probe to incorporate optical fibers for prostate USFM imaging. Although the fiber number may be limited due to the limited space, the transrectal transducer probe can be easily modified for USFM imaging. For example, in this particular system, 36 optical collection fibers can be used, as opposed to a single optical collection fiber used in conventional USFM systems, theoretically increasing the signal-to-noise ratio (SNR) by ˜6× (the square root relationship). However, in some instances a ˜4-5× increase in SNR and sensitivity is expected due to low amounts of spectral leakage. A computer controls the HIFU driving, focusing and scanning system. This computer also triggers a pulser/delay generator (PDG) with multiple channels to control the time sequence of firing the three pico-second (ps) pulsed lasers and the gating time of the three cooled-and-gated intensified charge-coupled devices (CG-ICCD). The specific time sequence is shown in FIG. 4b. The corresponding emission filters for the three fluorophores for purposes of this example are Colors 1-3 previously discussed above in EXAMPLE 1 (Color-1: 680-710 nm; Color-2: 740-770 nm and Color-3: >840 nm). However, these particular fluorophores are exemplary, and other fluorophores with different emission profiles can be used, such as those described below in Example 8.

Each laser will pair with one CG-ICCD camera (synchronized by the trigger signals from the PDG) for specifically exciting and detecting one fluorophore. The three laser-camera pairs are temporally separated for simultaneously detecting three fluorophores (see FIG. 4b). At each scanning point, USMF signals with three colors are acquired. By scanning the ultrasound focus, a USFM image with three colors can be acquired.

The high sensitivity of this system comes from at least the following strategies: (1) a large number of photon collection fibers (equivalent to spatial integration); (2) temporally blocks laser leakage and background fluorescence via the time gating method (suppressing noise); and (3) for each contrast agent a large number of gated emission pulses are temporally accumulated by the CG-ICCD (equivalent to temporal integration).

The high specificity originates from the capability of simultaneously resolving three fluorophores based on the following strategies: (1) three excitation lasers with well separated wavelengths; (2) the carefully designed emission filters to maximally match the emission spectra of the three fluorophores, but also maximally minimize the spectral leakage among the three fluorophores; and (3) three CG-ICCD cameras respectively synchronized with the three lasers for resolving the three fluorophores. In this design, each laser is synchronized with a specific CG-ICCD camera. Thus, each pair of the laser-and-camera can image one specific fluorophore. On the other hand, the three pairs of the laser-and-camera are temporally separated using the PDG to avoid overlap in time (cross talk). FIG. 4b schematically explains the principle how to differentiate the three fluorophores via the above strategies (in space, time and spectrum domains).

The ICCD camera usually has a slow response time (˜ms to hundreds of ms). However, the electronically controlled gate in front of the ICCD camera responds fast (˜ps) and the gate width is narrow (˜ns). Thus, the ICCD camera is turned on during the data acquisition at each location, but the gate is fast turned on and off repeatedly (at a level of tens of ns) to temporally select fluorescence photons for accumulating on the ICCD camera. Thus, each image acquired by the ICCD camera is the accumulation of many gated emission pulses.

### Example 4

### Estimation Algorithm of CSC Existence Probability at a Location

The absolute intensity of the USFM (fluorescence) signal depends on the excitation light distribution inside the tissue. Thus, tissue heterogeneity and the difference in light absorption and scattering at different wavelengths will affect USFM signals. To quantitatively deal with this issue, we define a normalized possibility function of P for each location based on three experimentally measurable parameters. This possibility function is independent of tissue heterogeneity and the optical difference at different wavelengths and can be used to quantify the possibility of CSCs existing at each location.

Equations 1-3 (shown below) represent three experimentally measured parameters acquired at Color-i (i=1, 2, or 3). (1) Φ_{BI}^{λ}^{ᵢ}: non-USFM background noise acquired before injection (BI) of USFM contrast agents, which consists of excitation light leakage (Φ_{Ex-Lek}^{λ}^{ᵢ}) and autofluorescence (Φ_{Auto-Fluo}^{λ}^{ᵢ}). 2) Φ_{AI-OFF}^{λ}^{ᵢ}: total background noise acquired after injection (AI) of USFM contrast agents, which includes non-USFM (Φ_{BI}^{λ}^{ᵢ}) and USFM background noise (Φ_{Non-100%-OFF-Fluo}^{λ}^{ᵢ}). The USFM background noise is mainly caused from the non-100%-OFF USFM contrast agents, which is independent of the location of the ultrasound transducer and can be calibrated to be similar for all three colors. (3) Φ_{ON}^{λ}^{ᵢ}: total data acquired when ultrasound is applied, which includes total background noise (Φ_{AI-OFF}^{λ}^{ᵢ}) and USFM signal (Φ_{USF}^{λ}^{ᵢ}).

Φ_{BI}^{λ}^{ᵢ}=(Φ_{Ex-Lek}^{λ}^{ᵢ}+Φ_{Auto-Fluo}^{λ}^{ᵢ})_{non-USFM-BG} Eq. (1)

Φ_{AI-OFF}^{λ}^{ᵢ}=(Φ_{Ex-Lek}^{λ}^{ᵢ}+Φ_{Auto-Fluo}^{λ}^{ᵢ})_{non-USFM-BG}+(Φ_{Non-100%-OFF-Fluo}^{λ}^{ᵢ})_{USFM-BG} Eq. (2)

Φ_{ON}^{λ}^{ᵢ}=(Φ_{Ex-Lek}^{λ}^{ᵢ}+Φ_{Auto-Fluo}^{λ}^{ᵢ})_{non-USFM-BG}+(Φ_{Non-100%-OFF-Fluo}^{λ}^{ᵢ})_{USFM-BG}+(Φ_{USF}^{λ}^{ᵢ})_{USFM- Signal} Eq. (3)

Qᵢ=(Φ_{ON}^{λ}^{ᵢ}−Φ_{AI-OFF}^{λ}^{ᵢ})/(Φ_{AI-OFF}^{λ}^{ᵢ}−Φ_{BI}^{λ}^{ᵢ})=(Φ_{USF}^{λ}^{ᵢ})_{USFM-Signal}/(Φ_{Non-100%-OFF- Fluo}^{λ}^{ᵢ})_{USFM-BG} Eq. (4)

Sᵢ=Qᵢ/(Q₁+Q₂+Q₃); Thus S₁+S₂+S₃=1 Eq. (5)

R(S₁,S₂)=S₁S₂S₃=S₁S₂(1−S₁−S₂) Eq. (6)

P(S₁,S₂)=Norm[R(S₁,S₂)]=R(S₁,S₂)/max [R(S₁,S₂)] Eq. (7)

By subtracting Eq. (2) from Eq. (3) and Eq. (1) from Eq. (2) and making the ratio between the two subtractions, Eq. (4) defines a new quantity Qᵢ, which describes the percentage of the USFM photons relative to the background USFM photons at a specific Color-i for each location. Thus, the optical difference at different wavelengths will be eliminated via the ratio (i.e. Qᵢ is a relative value). To eliminate the effect from tissue heterogeneity, Eq. (5) defines the 2^{nd} quantity (Sᵢ) that uses the sum of Qᵢ (i.e. Q₁+Q₂+Q₃) to rescale the percentage. Thus, the total sum of Sᵢ will be equal to 1 (Eq. (5)). Sᵢ is not affected by tissue heterogeneity (unlike Qᵢ). For example, if at one location the measurement shows (Q₁, Q₂, Q₃)=(0.4, 0.2, 0.2), it is calculated that (S₁,S₂, S₃)=(0.5, 0.25, 0.25). If at another location where the same CSCs exist but the excitation light fluence is 10 times lower, Qᵢ will be 10 times lower too (i.e. (Q₁, Q₂, Q₃)=(0.04, 0.02, 0.02)). This is because the numerator of Qᵢ depends on the location of the ultrasound but the denominator is independent of it. However, Sᵢ will remain the same, which means that Sᵢ independent of tissue heterogeneity.

To further quantify the possibility, Eq. (6) defines a function of R, which is the multiplication of the three Sᵢ (i.e. S₁, S₂,S₃). Because the sum of the three Sᵢ is equal to 1, the value of R is a function of S₁ and S₂ after S₃ is eliminated. Eq. (7) defines a possibility function of P(S₁,S₂), which is the normalized R(S₁,S₂). When S₁=S₂=S₃=⅓), the possibility of P=1, which means CSCs 100% exists. On the other hand, if any Sᵢ=0 (either S₁,S₂ or S₃), the possibility of P=0, which means no CSCs exists. Accordingly, the probability of the existence of a CSCs at each location can be determined based on the three measurable parameters and the calculated possibility function of P without effects from tissue heterogeneity and the different optical wavelengths (i.e. the Colors).

### Example 5

### Conjugation of USFM Contrast Agents onto Prostate CSCs

Prostrate CSCs lines PC3-KD and LAPC4-KD simultaneously overexpress CD44, CD133 and CD 117. A method of in vitro USFM imaging of these prostrate CSCs comprises incubating each cell line in a buffer solution having three different antibody-conjugated USFM contrast agents. The contrast agents are prepared using the procedures described in EXAMPLES 1 and 2, where the antibodies can be specific to CD44, CD133 and CD 117, and each antibody-conjugated contrast agent has a fluorophore of one of Color-1, Color-2, or Color-3. Conventional PC3 and/or LAPC4 cells can be used as control samples since CD44, CD133 and CD117 are not overexpressed or are not simultaneously overexpressed in these cells. The treated cells can be incubated for 5 hrs, 10 hrs, 20 hrs, 24 hrs, 48 hrs, 72 hrs, or 96 hrs. To remove the contrast agents introduced inside the cells due to the endocytosis, the treated cells can be incubated with a clear cell culture medium (no USFM contrast agents).

### Example 6

### Multi-Color USFM Imaging of Prostate CSCs in Tissue Scaffolds

A method of imaging prostate CSCs labeled with USFM contrast agents prepared in EXAMPLE 5 comprises injecting the labeled CSCs into hydrogel tissue scaffolds for imaging. The labeled CSCs can be imaged using the multi-color USFM thermometry system described in EXAMPLE 3. Furthermore the feasibility of imaging the three different colors (Color-1, Color-2, and Color-3) of the labeled CSCs can be compared with their control samples, and the probability of CSC existing at each location is estimated via the algorithm in EXAMPLE 4 for both CSC and non-CSC (i.e. control) samples.

The specificity to the USFM system towards identifying CSCs can be conducted by mixing CSCs with non-CSCs with the ratio of cells being 0, 0.1, 0.3, 0.7, 1, 3, 7, and 10. After mixing the cells, the mixture can be incubated in the three-color USFM contrast agent solution and similar processing described in EXAMPLE 5 can be conducted to remove unattached agents. The labeled and mixed cells can be imaged using the USFM system and the measured data can be processed using the defined probability function in EXAMPLE 4 for each location. The probability of detecting the CSCs increases as the ratio of CSC to non-CSC ratio increases.

### Example 7

### In-Vivo USFM Imaging of Centimeters Large Prostate CSCs in Live Mice

A method of in-vivo USFM imaging of centimeters large prostate CSCs in live mice comprises forming subcutaneous prostate tumors by injecting the PC3-KD or LAPC4-KD CSCs into SCID mice (NOD.CB17-Prkdc^{scid}). To image CSCs in tumors, the three contrast agents can be intravenously injected into a via tail veins. Two injection methods can be compared: (1) all injections at one time; (2) sequentially individual injection of each agent (see FIG. 8). The first method is simple but the three agents have some opportunities to stay together (such as in blood; interstitial fluid) to generate false signal. Appropriate time should be waited for washing out the free agents or background subtraction can be accomplished via a control method (see below). The second method can reduce the possibility of the three agent's simultaneous existence in background if the time interval between injections is appropriately selected.

After all the three agents are appropriately injected and the free agents are washed out, the tumors can be imaged by the USFM system with the three colors. To quantify the in vivo sensitivity of USFM imaging, the concentrations of the injected agents can be varied. To investigate the in vivo specificity, the results of simultaneous three-color imaging are compared with those of single- and/or two-color imaging (already available when finishing three-color imaging), and are further compared with the results of the control samples (described below). The results should be that the multi-color imaging has higher specificity to the CSCs compared with single-color imaging and the control sample imaging.

As a control, USFM contrast agents are not conjugated with targeting antibodies. Thus, the USFM images are considered background images. The images acquired from the targeting contrast agents stand out from these background images (see the concentration difference ΔC in FIG. 8).

The results can be statistically and quantitatively compared (between CSC and control samples; between single-color and multi-colors) and analyzed. Further validation can be conducted via immunofluorescence technology. Briefly, after all the experiments, the tumor tissue can be harvested and further processed for isolating CSCs from non-CSCs using the conventional technologies, such as flow cytometry technology, via the same biomarkers (note that only a portion of the biomarkers will be occupied by contrast agents during the in vivo USFM imaging). The collected CSC and non-CSC samples can be imaged again using the USFM system and conventional fluorescence microscopy for detecting the three colored USFM contrast agents for validation. All three colors can be detected from the CSC samples and show higher possibility value (i.e. higher P value) than those of non-CSC samples. The non-CSC samples lose around one or two probable P value.

In conclusion, the probability of CSCs occurring at different locations in a tumor can be imaged via the P value using Eq. (7). The probability of CSC occurring in CSC formed tumors is higher than that in non-CSC formed tumors. The three-color USFM can sensitively image CSCs and specifically differentiate CSCs from non-CSCs. The sensitivity and specificity to CSCs should be much higher than the existing imaging modalities, based on conventional data in literature. The resolution can be comparable with that of micro-MRI or micro-CT, and the imaging depth can be comparable with that of ultrasound.

### Example 8

### NIR-II USF Contrast Agents

In the previous Examples, multiplex imaging has been described for Colors 1-3, which have emission profiles in the NIR-I range. However, methods and compositions described herein are not limited solely to USF contrast agents having emission profiles in the NIR-I range. In some embodiments, NIR-II USF contrast agents can also be used.

For instance, an ICG-encapsulated poly(N-isopropylacrylamide) (“PNIPAM”) USF contrast agent was prepared having Ex=800 nm and Em=>915 nm. The ICG-encapsulated PNIPAM was prepared by as follows. N-isopropylacrylamide (NIPAM), allylamine (AM), sodium dodecyl sulfate (SDS), and N, N′-methylenebisacrylamide (BIS) were first dissolved with deionized water. ICG dye was quickly added into the reaction tube and stirred until fully dissolved. The solution was heated. The initiator, which was prepared by dissolving 4-4′-azobis(4-cyanopentanoic acid) (ACA) into water, was injected into the reaction tube. The resulting PNIPAM-AM-ICG nanoparticle solution was dialyzed against deionized water to afford purified ICG-encapsulated PNIPAM.

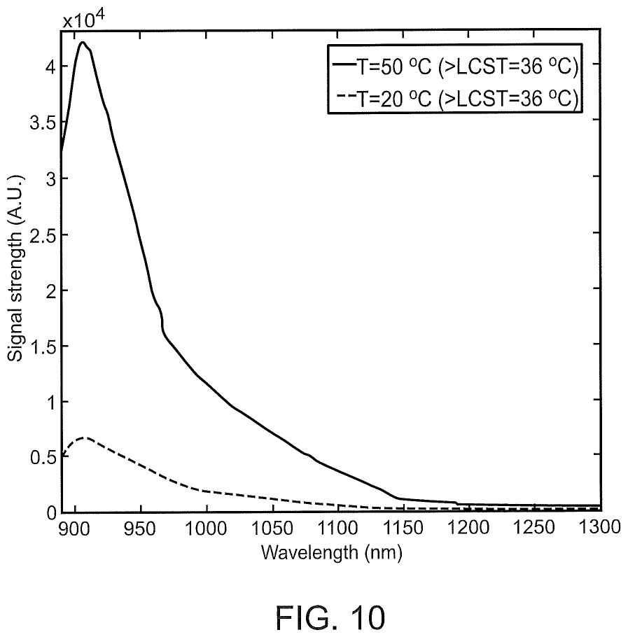

FIG. 10 demonstrates emission of the ICG-encapsulated PNIPAM at two different temperatures: one above the lower critical solution temperature (LCST) and the other above LCST. To image ICG-encapsulated PNIPAM USF contrast agent, a USF scan was conducted using a 800-nm excitation laser. As shown, the contrast agent displayed an emission tail of >915 nm at both temperatures, with the elevated temperature showing greater signal strength than the lower temperature. Thus, USF contrast agents having emissions in the NIR-II range have been demonstrated.

### Example 9

### USFM Imaging of NIR-II USF Contrast Agents

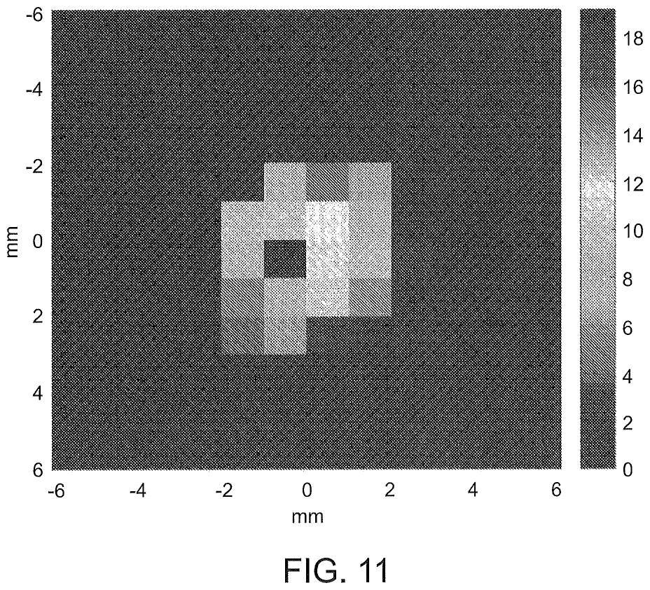

The USF imaging feasibility of NIR-II USF contrast agents was explored using the ICG-encapsulated PNIPAM contrast agent prepared in EXAMPLE 8. The ICG-encapsulated PNIPAM contrast agent was injected into a piece of porcine muscle at a depth of approximately 1 cm. The contrast agent was then imaged using the multi-color USFM thermometry system described in EXAMPLE 3. FIG. 11 shows an image of the emission of the ICG-encapsulated PNIPAM contrast agent. As evidenced by FIG. 11, the NIR-II contrast agent was resolvable in porcine muscle.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method of imaging cancer stem cells (CSCs) in a biological environment, the method comprising:
disposing a population of first ultrasound-switchable fluorophores having a first switching threshold in the biological environment, the first ultrasound-switchable fluorophores being functionalized for attachment to a first biomarker expressed by the CSCs;
disposing a population of second ultrasound-switchable fluorophores having a second switching threshold in the biological environment, the second ultrasound-switchable fluorophores being functionalized for attachment to a second biomarker expressed by the CSCs;
exposing the biological environment to an ultrasound beam to form an activation region within the biological environment;
disposing one or more of the first ultrasound-switchable fluorophores in the activation region to switch the first fluorophores from an off state to an on state;
disposing one or more of the second ultrasound-switchable fluorophores in the activation region to switch the second fluorophores from an off state to an on state;
exciting the first and second ultrasound-switchable fluorophores in the activation region with a beam of electromagnetic radiation; and
detecting light emitted by the first and second ultrasound-switchable fluorophores.

2. The method of claim 1, wherein the first or second ultrasound-switchable fluorophores comprises a fluorescent material having a peak emission wavelength between 680 nm and 710 nm.

3. The method of claim 1, wherein the first or second ultrasound-switchable fluorophores comprises a fluorescent material having a peak emission wavelength between 740 nm and 770 nm.

4. The method of claim 1, wherein the first or second ultrasound-switchable fluorophores comprises a fluorescent material having a peak emission wavelength that is longer than 800 nm.

5. The method of claim 1, wherein the first or second ultrasound-switchable fluorophores comprises a fluorescent material having an emission tail that is longer than 900 nm.

6. The method of claim 1, wherein the first ultrasound-switchable fluorophores emit light having a first average peak wavelength and the second ultrasound-switchable fluorophores emit light having a second average peak wavelength, and wherein the second average peak wavelength is 25-75 nm longer than the first average peak wavelength.

7. The method of claim 1, wherein exposing the biological environment to an ultrasound beam comprises scanning the biological environment with the ultrasound beam.

8. The method of claim 1, wherein the beam of electromagnetic radiation is in the near-infrared region (NIR) of the electromagnetic spectrum.

9. The method of claim 1, wherein the first and second ultrasound-switchable fluorophores in the activation region are excited by a single beam of electromagnetic radiation.

10. The method of claim 1, wherein the biological environment comprises tumor vasculature.

11. The method of claim 1, wherein the first and second ultrasound-switchable fluorophores comprise a thermo-sensitive polymer.

12. The method of claim 11, wherein the thermo-sensitive polymer comprises poly(N-isopropylacrylamide), a copolymer of N-isopropylacrylamide with one or more of acrylamide, N-tert-butylacrylamide, acrylic acid, and allylamine, or a polyoxypropylene-polyoxyethylene block copolymer.

13. The method of claim 1, further comprising:
disposing a population of third ultrasound-switchable fluorophores having a third switching threshold in the biological environment, the third ultrasound-switchable fluorophores being functionalized for attachment to a third bio-target expressed by the CSCs;
disposing one or more of the third ultrasound-switchable fluorophores in the activation region to switch the third fluorophores from an off state to an on state;
exciting the first, second, and third ultrasound-switchable fluorophores in the activation region with the beam of electromagnetic radiation; and
detecting light emitted by the first, second, and third ultrasound-switchable fluorophores.

14. The method of claim 13, wherein:
the first ultrasound-switchable fluorophores emit light having a first average peak wavelength;
the second ultrasound-switchable fluorophores emit light having a second average peak wavelength;
the third ultrasound-switchable fluorophores emit light having a third average peak wavelength; and
the third average peak wavelength is longer than each of the first and second average peak wavelengths.

15. The method of claim 13, wherein the light emitted by the first, second, and third pluralities of fluorophores is simultaneously detected.

16. The method of claim 13, wherein the first, second, and third switching thresholds are temperature thresholds.

17. A method of imaging a tumor comprising:
disposing first ultrasound-switchable fluorophores having a first switching threshold temperature in the tumor, the first ultrasound-switchable fluorophores being associated with a first emission spectrum;
disposing second ultrasound-switchable fluorophores having a second switching threshold temperature in the tumor, the second ultrasound-switchable fluorophores being associated with a second emission spectrum;
moving an ultrasound beam over a plurality of spatial locations within a plane of the tumor, wherein a temperature of the tumor within a focal zone of the ultrasound beam exceeds the first and second switching threshold temperatures thereby switching the first and second fluorophores in the focal zone from an off state to an on state;
exciting the first and second ultrasound-switchable fluorophores in the focal zone with a beam of electromagnetic radiation;
detecting light emitted by the first and second ultrasound-switchable fluorophores; and
characterizing a spatial location as a possible cancer stem cell (CSC) location in response to detecting light having peak wavelengths in each of the first and second emission spectra, and characterizing the spatial location as a non-CSC location in response to detecting light having a peak wavelength in only one of the first or the second emission spectrum.

18. The method of claim 17, wherein the plane of the tumor is located at a depth of 1-6 centimeters (cm) below a surface of a subject's skin.

19. The method of claim 17, wherein the first and second emission spectra are in the near-infrared region (NIR) of the electromagnetic spectrum.

20. The method of claim 17 further comprising generating a multi-colored image of the tumor, wherein spatial locations that correspond to emissions in the first emission spectra are correlated to a first color, spatial locations that correspond to emissions in the second emission spectra are correlated to a second color, and spatial locations that correspond to emissions being a combination of the first and second emission spectra are correlated to a third color.

21. The method of claim 17, wherein the tumor is located in a subject's breast, prostate, head, neck, throat, mouth, thyroid, skin, colon, cervix, or uterus.
